(19) United States
(12) Patent Application Publication
(10) Pub. No.: US 2009/0173352 A1
(43) Pub. Date: Jul. 9, 2009

(54) ON-OFF IMPLANT FOR SUPPORTING THE AIRWAY

(75) Inventors: Nikhil D. Bhat, Fremont, CA (US); Anant V. Hegde, Hayward, CA (US); Kasey Li, Palo Alto, CA (US); Matthew Goebel, Fairfax, CA (US); HongPeng Wang, Sunnyvale, CA (US); Ryan Woo, Fremont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pavad Medical, Fremont, CA (US)

(21) Appl. No.: 12/261,968

(22) Filed: Oct. 30, 2008

Related U.S. Application Data
(60) Provisional application No. 60/984,689, filed on Nov. 1, 2007.

(51) Int. Cl. A61F 5/56

(52) U.S. Cl. 128/848

(57) ABSTRACT

The present invention provides an airway implant device having an electroactive polymer element, including: a composite layer having a polymer substrate and a biocompatible conductive material, wherein the composite layer also can be opposing surfaces; and a conductive polymer layer disposed on at least one of the opposing surfaces of the composite layer, wherein the implant device is adapted and configured to modulate an opening of an air passageway. Some embodiments include a housing designed to conform to the shape of the palate. Some embodiments include an attachment element to secure the device to tissue. Methods of treating airway disorders such as sleep apnea and snoring with the airway implant device are disclosed herein.

### CROSS-REFERENCES TO RELATED APPLICATIONS

This application claims the benefit of U.S. Application No. 60/984,689, filed Nov. 1, 2007, the disclosure of which is incorporated herein by reference in its entirety.

### BACKGROUND OF THE INVENTION

Snoring is very common among mammals including humans. Snoring is a noise produced while breathing during sleep due to the vibration of the soft palate and uvula. Not all snoring is bad, except when it bothers the bed partner or others near the person who is snoring. If the snoring gets worst over time and goes untreated, it could lead to apnea.

Those with apnea stop breathing in their sleep, often hundreds of times during the night. Usually apnea occurs when the throat muscles and tongue relax during sleep and partially block the opening of the airway. When the muscles of the soft palate at the base of the tongue and the uvula relax and sag, the airway becomes blocked, making breathing labored and noisy and even stopping it altogether. Sleep apnea also can occur in obese people when an excess amount of tissue in the airway causes it to be narrowed.

In a given night, the number of involuntary breathing pauses or “apneic events” may be as high as 20 to 60 or more per hour. These breathing pauses are almost always accompanied by snoring between apnea episodes. Sleep apnea can also be characterized by choking sensations.

Sleep apnea is diagnosed and treated by primary care physicians, pulmonologists, neurologists, or other physicians with specialty training in sleep disorders. Diagnosis of sleep apnea is not simple because there can be many different reasons for disturbed sleep.

The specific therapy for sleep apnea is tailored to the individual patient based on medical history, physical examination, and the results of polysomnography. Medications are generally not effective in the treatment of sleep apnea. Oxygen is sometimes used in patients with central apnea caused by heart failure. It is not used to treat obstructive sleep apnea.

Nasal continuous positive airway pressure (CPAP) is the most common treatment for sleep apnea. In this procedure, the patient wears a mask over the nose during sleep, and pressure from an air blower forces air through the nasal passages. The air pressure is adjusted so that it is just enough to prevent the throat from collapsing during sleep. The pressure is constant and continuous. Nasal CPAP prevents airway closure while in use, but apnea episodes return when CPAP is stopped or it is used improperly. Many variations of CPAP devices are available and all have the same side effects such as nasal irritation and drying, facial skin irritation, abdominal bloating, mask leaks, sore eyes, and headaches.

Some versions of CPAP vary the pressure to coincide with the person's breathing pattern, and other CPAPs start with low pressure, slowly increasing it to allow the person to fall asleep before the full prescribed pressure is applied.

Dental appliances that reposition the lower jaw and the tongue have been helpful to some patients with mild to moderate sleep apnea or who snore but do not have apnea. A dentist or orthodontist is often the one to fit the patient with such a device.

Some patients with sleep apnea may need surgery. Although several surgical procedures are used to increase the size of the airway, none of them is completely successful or without risks. More than one procedure may need to be tried before the patient realizes any benefits. Some of the more common procedures include removal of adenoids and tonsils (especially in children), nasal polyps or other growths, or other tissue in the airway and correction of structural deformities. Younger patients seem to benefit from these surgical procedures more than older patients.

Uvulopalatopharyngoplasty (UPPP) is a procedure used to remove excess tissue at the back of the throat (tonsils, uvula, and part of the soft palate). The success of this technique may range from 30 to 60 percent. The long-term side effects and benefits are not known, and it is difficult to predict which patients will do well with this procedure.

Laser-assisted uvulopalatoplasty (LAUP) is done to eliminate snoring but has not been shown to be effective in treating sleep apnea. This procedure involves using a laser device to eliminate tissue in the back of the throat. Like UPPP, LAUP may decrease or eliminate snoring but not eliminate sleep apnea itself. Elimination of snoring, the primary symptom of sleep apnea, without influencing the condition may carry the risk of delaying the diagnosis and possible treatment of sleep apnea in patients who elect to have LAUP. To identify possible underlying sleep apnea, sleep studies are usually required before LAUP is performed.

Somnoplasty is a procedure that uses RF to reduce the size of some airway structures such as the uvula and the back of the tongue. This technique helps in reducing snoring and is being investigated as a treatment for apnea.

Tracheostomy is used in persons with severe, life-threatening sleep apnea. In this procedure, a small hole is made in the windpipe and a tube is inserted into the opening. This tube stays closed during waking hours and the person breathes and speaks normally. It is opened for sleep so that air flows directly into the lungs, bypassing any upper airway obstruction. Although this procedure is highly effective, it is an extreme measure that is rarely used.

Patients in whom sleep apnea is due to deformities of the lower jaw may benefit from surgical reconstruction, Surgical procedures to treat obesity are sometimes recommended for sleep apnea patients who are morbidly obese. Behavioral changes are an important part of the treatment program, and in mild cases behavioral therapy may be all that is needed. Overweight persons can benefit from losing weight. Even a 10 percent weight loss can reduce the number of apneic events for most patients. Individuals with apnea should avoid the use of alcohol and sleeping pills, which make the airway more likely to collapse during sleep and prolong the apneic periods. In some patients with mild sleep apnea, breathing pauses occur only when they sleep on their backs. In such cases, using pillows and other devices that help them sleep in a side position may be helpful.

Recently, Restore Medical, Inc., Saint Paul, Minn. has developed a new treatment for snoring and apnea, called the Pillar technique. Pillar System is a procedure where 2 or 3 small polyester rod devices are placed in the patient's soft palate. The Pillar System stiffens the palate, reduces vibration of the tissue, and prevents the possible airway collapse. Stiff implants in the soft palate, however, could hinder patient's normal functions like speech, ability to swallow, coughing and sneezing. Protrusion of the modified tissue into the airway is another long-term concern.

As the current treatments for snoring and/or apnea are not effective and have side-effects, there is a need for additional treatment options.

### BRIEF SUMMARY OF THE INVENTION

In one embodiment, the present invention provides an airway implant device having an electroactive polymer element, including: a composite layer having a polymer substrate and a biocompatible conductive material, wherein the composite layer also can be opposing surfaces; and a conductive polymer layer disposed on at least one of the opposing surfaces of the composite layer, wherein the implant device is adapted and configured to modulate an opening of an air passageway.

In a second embodiment, the present invention provides a method of controlling an opening of an air passageway, including: implanting an airway implant device proximal to an air passageway, in a wall of an air passageway or in both, the device having an electroactive polymer element including: a composite layer having a polymer substrate and a biocompatible conductive material, wherein the composite layer also can be opposing surfaces; and a conductive polymer layer disposed on at least one of the opposing surfaces of the composite layer, wherein the implant device is adapted and configured to modulate an opening of an air passageway; and energizing the electroactive polymer element for a fixed period of time, such that the electroactive polymer element adopts an energized state and maintains the energized state after the fixed period of time has passed, thereby completely or partially opening the air passageway.

In a third embodiment, the present invention provides a method of treating a disease using an airway implant device, including: implanting an airway implant device proximal to an air passageway or in a wall of an air passageway or in both, the device having an electroactive polymer element having: a composite layer having a polymer substrate and a biocompatible conductive material, wherein the composite layer also can be opposing surfaces; and a conductive polymer layer disposed on at least one of the opposing surfaces of the composite layer, wherein the implant device is adapted and configured to modulate an opening of an air passageway; and energizing the electroactive polymer element for a fixed period of time, such that the electroactive polymer element adopts an energized state and maintains the energized state after the fixed period of time has passed, thereby treating the disease.

For a further understanding of the nature and advantages of the invention, reference should be made to the following description taken in conjunction with the accompanying figures. It is to be expressly understood, however, that each of the figures is provided for the purpose of illustration and description only and is not intended as a definition of the limits of the embodiments of the present invention.

### BRIEF DESCRIPTION OF THE DRAWINGS

FIG. 1 illustrates one embodiment of the airway implant device.

FIG. 2 illustrates one embodiment of the airway implant device.

FIG. 3 illustrates one embodiment of the airway implant device.

FIG. 4 illustrates one embodiment of the airway implant device.

FIG. 5 illustrates a circuit diagram of an embodiment of the airway implant device.

FIG. 6 illustrates an embodiment of the airway implant device.

FIG. 7 illustrates a sectional view of an embodiment of the electroactive polymer element.

FIGS. 8A and 8B illustrates a sectional view of an embodiment of the electroactive polymer element.

FIG. 9 illustrates an embodiment of the electroactive polymer element.

FIG. 10 illustrates an embodiment of the electroactive polymer element.

FIG. 11 illustrates an embodiment of the electroactive polymer element.

FIG. 12 illustrates an embodiment of the electroactive polymer element.

FIG. 13 illustrates an embodiment of the electroactive polymer element.

FIG. 14 illustrates an embodiment of the electroactive polymer element.

FIG. 15 illustrates an embodiment of the electroactive polymer element.

FIG. 16 illustrates an embodiment of the electroactive polymer element.

FIG. 17 illustrates an embodiment of the electroactive polymer element.

FIG. 18 illustrates an embodiment of the electroactive polymer element.

FIG. 19 illustrates an embodiment of the electroactive polymer element.

FIG. 20 illustrates an embodiment of the implanted portion of the airway implant device.

FIG. 21 illustrates an embodiment of the airway implant device.

FIG. 22 illustrates an embodiment of the non-implanted portion in the form of a mouthpiece.

FIG. 23 illustrates an embodiment of the non-implanted portion in the form of a mouthpiece.

FIG. 24 illustrates an embodiment of the non-implanted portion.

FIG. 25 shows a sagittal section through a head of a subject illustrating an embodiment of a method for using the airway implant device.

FIG. 26 illustrates an anterior view of the mouth with see-through mouth roofs to depict an embodiment of a method for using the airway implant device.

FIG. 27 illustrates an anterior view of the mouth with see-through mouth roofs to depict an embodiment of a method for using the airway implant device.

FIG. 28 illustrates an anterior view of the mouth with see-through mouth roofs to depict an embodiment of a method for using the airway implant device.

FIG. 29 illustrates an anterior view of the mouth with see-through mouth roofs to depict an embodiment of a method for using the airway implant device.

FIG. 30 illustrates an embodiment of an inductive coupling system associated with the airway implant device.

FIG. 31 illustrates an embodiment of the airway implant device.

FIG. 32 illustrates an embodiment of the airway implant device.

FIG. 33 illustrates an embodiment in which a patient wears the non-implanted portion of the device on the cheeks.

FIG. 34A-34B illustrates an embodiment of a method of the invention with the airway implant in the soft palate.

FIG. 35A-35B illustrates an embodiment of a method of the invention with the airway implants in the soft palate and lateral pharyngeal walls.

FIG. 36A-36B illustrates an embodiment of a method of the invention with the airway implants in the lateral pharyngeal walls.

FIG. 37 depicts an embodiment of an airway implant device.

FIGS. 38A and 38B depict an embodiment of an airway implant device.

FIGS. 39A, 39B, and 39C illustrate terms used in describing the anatomy of a patient and orientation attributes of the invention.

FIG. 40A illustrates an embodiment of the airway implant device.

FIG. 40B illustrates the airway implant device of FIG. 40A, viewed from the anterior side of the implant, looking toward the posterior end, wherein the implant device is implanted in the palate.

FIG. 41A illustrates an embodiment of the airway implant device.

FIG. 41B illustrates the airway implant device of FIG. 41A, viewed from the anterior side of the implant, looking toward the posterior end, wherein the implant device is implanted in the palate.

FIG. 42A illustrates an embodiment of the airway implant device with a T-shaped attachment element.

FIG. 42B illustrates an embodiment of the airway implant device with a perforated attachment element.

FIGS. 43A and 43B illustrates an embodiment of the airway implant device with saw-blade like directional attachment element.

FIG. 44 illustrates an embodiment of the airway implant device with power connecting element.

FIG. 45 illustrates an embodiment of the airway implant system with both an implantable device and a non-implantable wearable element.

FIG. 46A illustrates an isometric view of the wearable element.

FIG. 46B illustrates a bottom view of the wearable element.

FIG. 47 illustrates a cross-sectional view of the airway implant system in the patient soft palate.

FIG. 48 depicts an embodiment of an airway implant device.

FIG. 49 is a simplified schematic drawing of an exemplary tongue implant device in accordance with another embodiment of the present invention.

FIGS. 50A-D illustrate one exemplary procedure for the placement of the tongue implant.

FIGS. 51A and B illustrate two exemplary wire configurations of the device.

FIG. 52 shows a schematic of two embodiments of the device.

FIG. 53 shows a schematic of the device having a staggered polypyrrole coating.

FIG. 54 illustrates the effect of using a silicone coating on the electromechanical life cycle of the polypyrrole actuators, using a 1.2V, 1 minute actuation and 2 minute rest period.

FIG. 55 illustrates the effect on electromechanical life cycle of the polypyrrole actuators using an 8 hour holding test cycle with 1.2V and 40 μAhr capacity control method.

FIG. 56 shows several embodiments of the conductive polymer layer patch coating the composite layer. FIG. 56A shows the conductive polymer completely coating each opposing surface of the composite layer. FIG. 56B shows the conductive polymer completely coating one of the opposing surfaces of the composite layer and patch coating the other opposing surface. FIG. 56C shows each opposing surface of the composite layer patch coated with the conductive polymer layer, such that one opposing surface has two areas coated with the conductive polymer layer, and the other opposing surface has three areas coated with the conductive polymer later. FIG. 56D shows each opposing surface of the composite layer patch coated with the conductive polymer layer, such that one opposing surface has two areas coated with the conductive polymer layer, and the other opposing surface has four areas coated with the conductive polymer later. FIG. 56E shows each opposing surface of the composite layer patch coated with the conductive polymer layer, such that each opposing surface has three areas coated with the conductive polymer layer, but one opposing surface has a greater surface area of the opposing surface with the conductive polymer later. In other embodiments, a greater or smaller number of areas of each opposing surface of the composite layer can be patch coated with the conductive polymer layer.

### DETAILED DESCRIPTION OF THE INVENTION

### I. General

The present invention provides an airway implant device including an electroactive polymer element that is flexible in a non-energized state and that is stiff in an energized state. The electroactive polymer element does not require constant power in order to maintain the energized state, and can maintain the energized state even when the power to the electroactive polymer element is turned off. In this fashion, a subject using the airway implant device of the present invention need only provide power for a short time to the electroactive polymer element, thus avoiding having to wear a power source throughout the night.

### II. Airway Implant Device

In some embodiments, the present invention provides an airway implant device including an electroactive polymer element having a composite layer having a polymer substrate and a biocompatible conductive material, wherein the composite layer also can be opposing surfaces; and a conductive polymer layer disposed on at least one of the opposing surfaces of the composite layer, wherein the implant device is adapted and configured to modulate an opening of an air passageway.

A first aspect of the invention is a device for the treatment of disorders associated with improper airway patency, such as snoring or sleep apnea. The device can be an actuator element to adjust the opening of the airway. In a preferred embodiment, the actuator element can be an electroactive polymer (EAP) element. The electroactive polymer element in the device assists in maintaining appropriate airway opening to treat the disorders. Typically, the EAP element provides support for the walls of an airway, when the walls collapse, and thus, completely or partially opens the airway.

The device functions by maintaining energized and non-energized configurations of the EAP element. In preferred embodiments, during sleep, the EAP element is energized with electricity to change its shape and thus modify the opening of the airway. Typically, in the non-energized configuration the EAP element is flexible and in the energized configuration is less flexible. The EAP element of the device can have a pre-set non-energized configuration wherein it is substantially similar to the geometry of the patient's airway where the device is implanted.

In some embodiments, the device, in addition to the EAP element, can be an implantable receiver in electrical communication with the EAP element. A conductive lead connects the EAP element and the implantable receiver to each other. The device of the present invention typically can be a power source in electrical communication with the EAP element and/or the implantable receiver, such as a battery or a capacitor. The battery can be disposable or rechargeable.

Preferred embodiments of the invention include a non-implanted portion, such as a mouthpiece, to control the implanted EAP element. The mouthpiece is typically in conductive or inductive communication with an implantable receiver. In one embodiment, the mouthpiece is a dental mouthpiece with an induction coil and a power source. The dental mouthpiece can also include a pulse-width-modulation circuit. When a dental mouthpiece is used it is preferably custom fit for the individual biological subject. If the implantable receiver is in inductive communication, it will typically include an inductive receiver, such as a coil. The implantable receiver can also include a conductive receiver, such as a dental filling, a dental implant, an implant in the oral cavity, an implant in the head or neck region. In one embodiment, the device can be a dermal patch with a coil, circuit and power source, in communication with the implantable receiver. The dermal patch can also include a pulse-width-modulation circuit.

Another aspect of the invention is a method to modulate air flow through airway passages. Such modulation is used in the treatment of diseases such as snoring and sleep apnea. One method of the invention is a method for modulating the airflow in airway passages by implanting in a patient a device having an actuator element and controlling the device by energizing the actuator element. The actuator element preferably can be an electroactive polymer element. The actuator element can be controlled with a mouthpiece inserted into the mouth of the patient. The energizing is typically performed with the use of a power source in electrical communication, either inductive communication or conductive communication, with the actuator element. A receiver can be used to energize the actuator element by placing it in electrical communication with the power source. Depending on the condition being treated, the actuator element is placed in different locations such as soft palate, airway sidewall, uvula, pharynx wall, trachea wall, larynx wall, a tongue and/or nasal passage wall.

A preferred embodiment of the device of the present invention can be an implantable actuator element; an implantable receiver; an implantable lead wire connecting the actuator element and the receiver; a removable receiver; and a removable power source; wherein the actuator element can be an electroactive polymer element.

In some embodiments, the device of the present invention also can be an anode, a cathode, a first inductor, a controller and a non-implanted portion. In some other embodiments, the non-implanted portion can be a mouthguard, a power supply and a second inductor. In still other embodiments, the first inductor and the second inductor are configured to interact. In yet other embodiments, the electroactive polymer element also can have wires for connection with the first inductor. In still yet other embodiments, the electroactive polymer element is configured for implantation into a soft palate, a lateral pharyngeal wall, a tongue or combination thereof.

In another embodiment, the present invention provides a device that also can be a coating to prevent or promote tissue growth. In other embodiments, the device also can be a coating of polypropylene, poly-L-lysine, poly-D-lysine, polyethylene glycol, polyvinyl alcohol, polyvinyl acetate, polymethyl methacrylate, hyaluronic acid and combinations thereof.

In a further embodiment, the airway implant device is controlled by an inductive coupling mechanism.

### III. Electroactive Polymer Element

The electroactive polymer element of the present invention can be a composite layer and a conductive polymer layer. The composite layer of the present invention can be a polymer substrate and a biocompatible conductive material.

Electroactive polymer is a type of polymer that responds to electrical stimulation by physical deformation, change in tensile properties, and/or change in hardness. There are several types of electroactive polymers like dielectric electrostrictive polymers, conducting polymers, ion exchange polymers and ion exchange polymer metal composites (IPMC). The particular type of EAP used in the making the disclosed device can be any of the aforementioned electroactive polymers.

A. Composite Layer

The composite layer of the present invention can be a polymer substrate and a biocompatible conductive material.

1. Polymer Substrate

Polymer substrates useful in the device of the present invention can be any suitable polymer material. Suitable materials for the polymer substrate portion of the electroactive polymer element include, but are not limited to, an ion exchange polymer, an ion exchange polymer metal composite, an ionomer base material. In some embodiments, the polymer substrate is perfluorinated polymer such as polytetrafluoroethylene, polyfluorosulfonic acid, perfluorosulfonate, and polyvinylidene fluoride. Other suitable polymers include polyethylene, polypropylene, polystyrene, polyaniline, polyacrylonitrile, cellophane, cellulose, regenerated cellulose, cellulose acetate, polysulfone, polyurethane, polyvinyl alcohol, polyvinyl acetate, polyvinyl pyrrolidone.

In some embodiments, the polymer substrate can be polytetrafluoroethylene, polyfluorosulfonic acid, perfluorosulfonate, polyvinylidene fluoride, polyethylene, polypropylene, polystyrene, polyaniline, polyacrylonitrile, cellulose, regenerated cellulose, cellulose acetate, polysulfone, polyurethane, polyvinyl alcohol, polyvinyl acetate, polyvinyl pyrrolidone, polymethyl methacrylate, silicon and combinations thereof. In some other embodiments, the polymer substrate can be polyurethane. One of skill in the art will appreciate that other materials are useful as the polymer substrate of the present invention.

Suitable shapes of the composite layer include three dimensional shape, substantially rectangular, substantially triangular, substantially round, substantially trapezoidal, a flat strip, a rod, a cylindrical tube, an arch with uniform thickness or varying thickness, a shape with slots that are perpendicular to the axis, slots that are parallel to the longitudinal axis, a coil, perforations, and/or slots.

IPMC is a polymer and metal composite that uses an ionomer as the base material. Ionomers are types of polymers that allow for ion movement through the membrane. There are several ionomers available in the market and some of the suited ionomers for this application are polyethylene, polystyrene, polytetrafluoroethylene, polyvinylidene fluoride, polyfluorosulfonic acid based membranes like NAFION® (from E.I. Du Pont de Nemours and Company, Wilmington, Del.), polyaniline, polyacrylonitrile, cellulose, cellulose acetates, regenerated cellulose, polysulfone, polyurethane, or combinations thereof. A conductive metal, for example gold, silver, platinum, palladium, copper, carbon, or combinations thereof, can be deposited on or embedded in the ionomer to make the IPMC. The IPMC element can be formed into many shapes, for example, a strip, rod, cylindrical tube, rectangular piece, triangular piece, trapezoidal shape, arch shapes, coil shapes, or combinations thereof. The IPMC element can have perforations or slots cut in them to allow tissue in growth.

2. Biocompatible Conductive Material

The device of the present invention can be any suitable biocompatible conductive material. Biocompatible conductive material useful in the present invention can be, but is not limited to, metals, including metal alloys and metal oxides, ceramics, conducting polymers and conductive carbon, such as graphite and graphite-like carbon materials.

Metals useful in the present invention include the alkali metals, alkali earth metals, transition metals and post-transition metals. Alkali metals include Li, Na, K, Rb and Cs. Alkaline earth metals include Be, Mg, Ca, Sr and Ba. Transition metals include Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg and Ac. Post-transition metals include Al, Ga, In, Tl, Ge, Sn, Pb, Sb, Bi, and Po. One of skill in the art will appreciate that the metals described above can each adopt several different oxidation states, all of which are useful in the present invention. In some instances, the most stable oxidation state is formed, but other oxidation states are useful in the present invention. In addition, several metals can be mixed together to form an alloy, such as brass and steel.

In some embodiments, the biocompatible conductive material is platinum, gold, silver, palladium, copper, and/or carbon. In some other embodiments, the biocompatible conductive material is conductive carbon, Ag, Au, Cu, Pt, Pd, Rh, Ir, Ru, Os and Re. In still other embodiments, the biocompatible conductive material can be Pt. In other embodiments, the composite layer can be polyurethane and Pt.

B. Conductive Polymer Layer

The conductive polymer layer can be any conducting polymer. Conducting polymers useful as the conductive polymer layer of the instant invention include, but are not limited to, poly(acetylene)s, poly(pyrrole)s, poly(thiophene)s, poly(aniline)s, poly(fluorene)s, poly(3-alkylthiophene)s, polytetrathiafulvalenes, polynaphthalenes, poly(p-phenylene sulfide), and poly(para-phenylene vinylene)s. In some embodiments, the conductive polymer layer can be polypyrrole, polyaniline and polyacetylene. In some other embodiments, the conductive polymer layer can be polypyrrole.

In some embodiments, the conductive polymer layer includes a copolymer. The copolymer can include any conductive polymer, such as those described above. In other embodiments, the copolymer is prepared using at least two of the following comonomers: pyrrole, 3,4-ethylene-dioxythiophene, 4-(3-pyrrolyl)-butyric acid, 3-methylpyrrole, 1H-pyrrole-1-propanoic acid, 1-(phenylsulfonyl)pyrrole, N-methylpyrrole, 1H-pyrrole-3-methyl carboxylate, N-benzylpyrrole, 4-(1H-pyrrol-1-yl)benzoic acid and 3-acetyl-1-methylpyrrole. In some other embodiments, the copolymer includes pyrrole and N-methylpyrrole. The copolymer can be a block copolymer, an alternating copolymer or a random copolymer. Other types of copolymers are also useful in the present invention. The copolymers can include comonomers at a variety of relative amounts. In some embodiments, the ratio of the monomers can be 100:1, 50:1, 25:1, 20:1, 15:1, 10:1, 5:1, 4:1, 3:1, 2:1 or 1:1. Other ratios of the monomers are useful in the present invention.

In other embodiments, the conductive polymer layer can include a dopant. Dopants useful in the conductive polymer layer include, but are not limited to, metals, ceramics and salts. In some embodiments, the dopants can be ionic dopants having mobile cations or anions, such as metals, ammonium salts, carboxylates, phosphate and sulfonates. In some other embodiments, the ionic dopant can be a biocompatible ionic dopant. In still other embodiments, the biocompatible ionic dopant can be a salt including Na⁺ ions. In another embodiment, the dopant can be dodecyl benzenesulfonic acid sodium salt. Other dopants useful in the present invention include, but are not limited to, Li⁺, tetrabutylammonium (TBA⁺), K⁺, PF₆—, trifluoromethanesulfonamide (TFSI⁻), polystyrenesulphonate (PSS⁻), tetrafluoroborate (TFB⁻) and CF₃SO₃⁻.

In some embodiments, the conductive polymer layer can be polypyrrole doped with dodecyl benzenesulfonic acid. In other embodiments, the electroactive polymer element can be a composite of polyurethane and Pt, and polypyrrole doped with dodecyl benzenesulfonic acid. In some other embodiments, the electroactive polymer element can be two conductive polymer layers each deposited on one of the opposing faces of the composite layer. In still other embodiments, at least one of the opposing surfaces is patch coated with one of the conductive polymer layers. In yet other embodiments, both of the opposing surfaces are patch coated with the conductive polymer layers.

In another embodiment, the present invention provides a device having a plurality of composite layers and a plurality of conductive polymer layers in alternating layers such that each conductive polymer layer is disposed on an opposing face of one of the composite layers.

C. Additional Components

The electroactive polymer element can also include a silicone rubber coating. For example, the silicone rubber coating can coat all of or portions of the composite layer. The silicone coating can coat only the composite layer, or both the composite layer and the conductive polymer layer. In addition, the silicone coating can coat none of the conductive polymer layer, a portion of, or all of the conductive polymer layer.

The electroactive polymer element has, in some embodiments, multiple layers of the electroactive polymer with or without an insulation layer separating the layers of the electroactive polymer. Suitable insulation layers include, but are not limited to, silicone, polyurethane, polyimide, nylon, polyester, polymethylmethacrylate, polyethylmethacrylate, neoprene, styrene butadiene styrene, or polyvinyl acetate.

In some embodiments, the actuator element, the entire device, or portions of the airway implant have a coating. The coating isolates the coated device from the body fluids and/or tissue either physically or electrically. The device can be coated with polypropylene and polyvinylidene fluoride to minimize tissue growth, or with poly-L-lysine, poly-D-lysine, polyethylene glycol, polyvinyl alcohol, polyvinyl acetate, hyaluronic acid, and/or methylmethacrylate to promote tissue growth.

In other embodiments, the electroactive polymer element also includes an ion source disposed on one of the opposing surfaces of the composite layer. The ion source of the present invention can be a salt, such as sodium chloride, phosphonic acid sodium salt or sulfonic acid sodium salt. The ion source can be mixed in a gel electrolyte, like agar gel, polyvinyl alcohol etc. Ions useful as the ion source include, but are not limited to, lithium, sodium, potassium, ammonium, magnesium and calcium. Other ions are useful in the electroactive polymer element of the present invention. In some embodiments, the dopant in the conductive polymer layer is a salt having a sodium counterion. In other embodiments, the dopant in the conductive polymer layer has the same counterion as the ion of the ion source. In some other embodiments, the ion source is a sodium ion source.

### IV. Methods of Making Electroactive Polymer Element

The electroactive polymer element includes both a composite layer and a conductive polymer layer.

In some embodiments, the composite layer is an IPMC strip which is made from a polymer substrate base material of an ionomer sheet, film or membrane. The ionomer sheet is formed using ionomer dispersion. IPMC is made from the base ionomer of, for example, polyethylene, polystyrene, polytetrafluoroethylene, polyvinylidene fluoride (PVDF) (e.g., KYNAR® and KYNAR Flex®, from ATOFINA, Paris, France, and SOLEF®, from Solvay Solexis S.A., Brussels, Belgium), hydrophilic-PVDF (h-PVDF), polyfluorosulfonic acid based membranes like NAFION® (from E.I. Du Point de Nemours and Company, Wilmington, Del.), polyaniline, polyacrylonitrile, cellulose, cellulose acetates, regenerated cellulose, polysulfone, polyurethane, and combinations thereof.

The polymer substrate can be any material, such as those described above. The polymer substrate can be coated or embedded with the biocompatible conductive material. In some embodiments, the biocompatible conductive material is in the form of wires or particles.

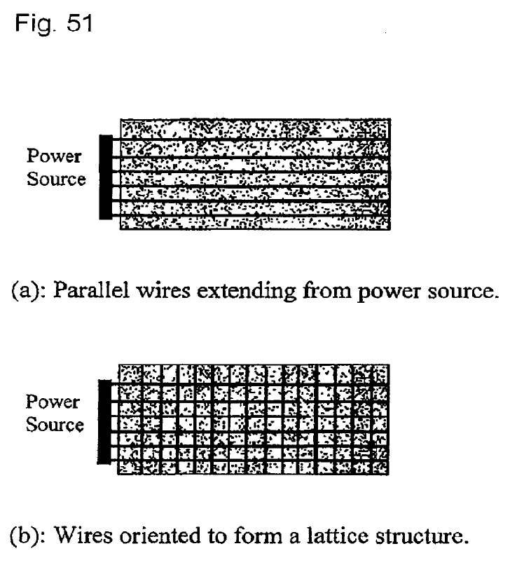

When the biocompatible conductive material is in the form of wires, the wires can have any thickness and pitch. In some embodiments, the wires have a pitch of from about 1 μm to about 1 mm. In addition, the wires can be in any configuration, such as parallel, lattice, zig-zag, etc. (see FIGS. 51A and 51B).

When the biocompatible conductive material is in the form of particles, the particles can be of any size and shape. In some embodiments, the particles are from about 0.1 μm to about 100 μm in size.

A. Coating the Polymer Substrate with the Biocompatible Conductive Material

In some embodiments, the polymer substrate is coated with the biocompatible conductive material.

The conductive material that is deposited on the polymer substrate can be gold, platinum, silver, palladium, copper, graphite, conductive carbon, or combinations thereof. Conductive material is deposited on the polymer substrate either by electrolysis process, vapor deposition, sputtering, electroplating, spraying, coating, dipping, brushing or combination of processes.

In some embodiments, the composite layer is an IPMC strip which is made from a polymer substrate base material of an ionomer sheet, film or membrane. The IPMC can be cut into the desired implant shape for the EAP element. The electrical contact (e.g., anode and cathode wires for EAP element) can be connected to the EAP surfaces by, for example, soldering, welding, brazing, potting using conductive adhesives, or combinations thereof. The EAP element is configured, if necessary, into specific curved shapes using mold and heat setting processes.

In some embodiments, the EAP element is insulated with electrical insulation coatings. Also, the EAP element can be insulated with coatings that promote cell growth and minimize fibrosis, stop cell growth, or kill nearby cells. The insulation can be a biocompatible material. The EAP element is coated with polymers such as polypropylene, poly-L-lysine, poly-D-lysine, polyethylene glycol, polyvinyl alcohol, polyvinyl acetate, polymethyl methacrylate, or combinations thereof. The EAP element can also be coated with hyaluronic acid.

The coating is applied to the device by standard coating techniques like spraying, electrostatic spraying, brushing, vapor deposition, dipping, etc. In one example, a perfluorosulfonate ionomer, PVDF or h-PVDF sheet is prepared for manufacturing the EAP element. In an optional step, the sheet is roughened on both sides using, for example, about 320 grit sand paper and then about 600 grit sand paper; then rinsed with deionized water; then submerged in isopropyl alcohol (IPA); subjected to an ultrasonic bath for about 10 minutes; and then the sheet is rinsed with deionized water. The sheet is boiled for about 30 minutes in hydrochloric acid (HCl). The sheet is rinsed and then boiled in deionized water for about 30 minutes.

The sheet is then subject to ion-exchange (i.e., absorption). The sheet is submerged into, or otherwise exposed to, a metal salt solution at room temperature for more than about three hours. Examples of the metal salt solution are tetraammineplatinum chloride solution, silver chloride solution, hydrogen tetrachloroaurate, tetraamminepalladium chloride monohydrate or other platinum, gold, silver, carbon, copper, or palladium salts in solution. The metal salt solution typically has a concentration of greater than or equal to about 200 mg/100 ml water. 5% ammonium hydroxide solution is added at a ratio of 2.5 ml/100 ml to the tetraammineplatinum chloride solution to neutralize the solution. The sheet is then rinsed with deionized water. Primary plating is then applied to the sheet. The sheet is submerged in water at about 40° C. 5% solution by weight of sodium borohydride and deionized water is added to the water submerging the sheet at 2 ml/180 ml of water. The solution is stirred for 30 minutes at 40° C. The sodium borohydride solution is then added to the water at 2 ml/180 ml of water and the solution is stirred for 30 minutes at 40° C. This sodium borohydride adding and solution stirring is performed six times total. The water temperature is then gradually raised to 60° C. 20 ml of the sodium borohydride solution is then added to the water. The solution is stirred for about 90 minutes. The sheet is then rinsed with deionized water, submerged into 0.1N HCl for an hour, and then rinsed with deionized water.

In some embodiments, the sheet receives a second plating. The sheet is submerged or otherwise exposed to a tetraammineplatinum chloride solution at a concentration of about 50 mg/100 ml deionized water. 5% ammonium hydroxide solution is added at a rate of 2 ml/100 ml of tetrammineplatinum chloride solution. 5% by volume solution of hydroxylamine hydrochloride in deionized water is added to the tetraammineplatinum chloride solution at a ratio of 0.1 of the volume of the tetraammineplatinum chloride solution. 20% by volume solution of hydrazine monohydrate in deionized water is added to the tetraammineplatinum chloride solution at a ratio of 0.05 of the volume of the tetraammineplatinum chloride solution. The temperature is then set to about 40° C. and the solution is stirred.

A 5% solution of hydroxylamine hydrochloride is then added at a ratio of 2.5 m/100 ml of tetraammineplatinum chloride solution. A 20% solution of hydrazine monohydrate solution is then added at a ratio of 1.25 ml/100 ml tetraammineplatinum chloride solution. The solution is stirred for 30 minutes and the temperature set to 60° C. The above steps in this paragraph can be repeated three additional times. The sheet is then rinsed with deionized water, boiled in HCl for 10 minutes, rinsed with deionized water and dried.

In some embodiments, the polymer base is dissolved in solvents, for example dimethyl acetamide, acetone, methylethyl ketone, toluene, dimethyl carbonate, diethyl carbonate, and combinations thereof. The solvent is then allowed to dry, producing a thin film. While the solution is wet, a low friction, (e.g., glass, Teflon) plate is dipped into the solution and removed. The coating on the plate dries, creating a thick film. The plate is repeatedly dipped into the solution to increase the thickness of the film.

Polyvinyl alcohol, polyvinyl pyrrolidone, polyvinyl acetate or combinations thereof can be added to a PVDF solution before drying, thus contributing hydrophilic properties to PVDF and can improve ion migration through the polymer film during manufacture. Dye or other color pigments can be added to the polymer solution.

B. Embedding the Polymer Substrate with the Biocompatible Conductive Material

In some embodiments, the composite layer includes polymer substrate embedded with the biocompatible conductive material. The amount of biocompatible material embedded in the polymer substrate can be any amount, such as 0.1:1 (w/w), 0.2:1, 0.3:1, 0.4:1, 0.5:1, 0.6:1, 0.7:1, 0.8:1, 0.9:1, 1.0:1, 1.5:1, 2:1, 2.5:1, 3:1, 3.5:1, 4:1, 4.5:1, 5:1, 6:1, 7:1, 8:1, 9:1, 10:1. In some embodiments, the ratio of biocompatible conductive material to polymer substrate is from about 0.1:1 (w/w) to about 5:1 (w/w). In other embodiments, the ratio of biocompatible conductive material to polymer substrate is about 2:1 (w/w).

When the biocompatible conductive material is embedded in the polymer substrate, the biocompatible conductive material can be in particulate form. The particles of biocompatible conductive material can be from 0.1 microns to 100 microns, preferably from 0.5 to 10 microns. The particles of biocompatible conductive material can adopt any useful shape, such as spherical, pyramid, rod etc.

The composite layer of the present invention can be made using any suitable materials described above. For example, the composite layer having Pt particles embedded in polyurethane can be prepared from the following procedure. Mix 2.5 g of polyurethane with 50 ml of N,N-Dimethylacetimide (DMAc) solvent. Stir for 120 minutes or until the polyurethane is completely dissolved. Add 5.0 g of conductive powder (Pt) to the solution. Stir till completely mixed. Then cast films with this solution. Keep in oven for some time till solvent is evaporated. Remove the casted film. Measure surface conductivity.

C. Preparation of the Conductive Polymer Layer

The conductive polymer layer can be prepared using a variety of methods. A platinum wire lattice was applied to one side of a 0.005 inch polyurethane substrate. Platinum particles were then brushed on. A piece of gold foil was used to make electrical contact with the platinum wires at the end of the sample. The end of the sample was then covered with 3M VHB acrylic tape, leaving some gold foil exposed, in order to mask the atmosphere/PPy (DBS) solution interface. Polypyrrole (PPy) doped with dodecyl benzene sulfonic acid (DBS) was grown on the substrate in a solution of 0.2 M PPy, 0.2M DBS in distilled water with a constant 3 mA applied current. Current was removed after six hours when the polypyrrole had achieved 100% coverage of the specimen.

The conductive polymer layer can also be prepared on the composite layer by embedding biocompatible conductive particles in the polymer substrate. A base layer with two composite layers separated by an insulating layer was used as the working electrode in a two electrode electrochemical cell. Two stainless steel plates facing the two conductive composite layers were used as the counter electrode. Polypyrrole (PPy) doped with dodecyl benzene sulfonic acid (DBS) was grown on the substrate in a solution of pyrrole monomer varying from 1×10⁻³ M to 5×10⁻¹ M concentration with DBS varying from 1×10⁻³ M to 5×10⁻¹ M concentration in distilled water with a constant 3 mA applied current. A constant voltage between 1 V and 3 V was applied for 2 to 240 hrs or until the target polypyrrole thickness had been achieved.

When the conductive polymer layer includes a copolymer, such as those described above, the comonomers are mixed and polymerized according to the procedure described above. For example, a copolymer of pyrrole and N-methylpyrrole can be prepared by mixing pyrrole, N-methylpyrrole and, optionally, DBS. In some embodiments, the conductive polymer layer includes a copolymer of pyrrole and N-methylpyrrole doped with dodecyl benzenesulfonic acid.

The conductive polymer layer can coat the composite layer in a variety of configurations. For example, the conductive polymer layer can coat at least one of the opposing surfaces of the composite layer. In addition, when coating one of the opposing surfaces of the composite layer, the conductive polymer layer can fully or partially coat the opposing surface. When one of the opposing surfaces of the composite layer is partially coated by the conductive polymer layer, the conductive polymer layer can be coated in patches; stripes that can be oriented along the length of the device, orthogonal to the length of the device, or diagonally; a checkerboard pattern; or others (see FIG. 53).

Each opposing surface of the composite layer can be coated with the conductive polymer layer separately and in a configuration different from, or the same as, the other opposing surface. For example, one opposing surface of the composite layer can be completely coated with the conductive polymer layer, and the other opposing surface can be patch coated with the conductive polymer layer. In addition, the pitch of the patch coating of the conductive polymer layer can be the same or different for the opposing surfaces.

The conductive polymer layer can be patch coated onto the composite layer by a variety of methods known in the art. For example, the composite layer can be coated with a blocking agent in order to prevent growth of the conductive polymer layer where the blocking agent is coated. When the polymerization conditions described above are used, the conductive polymer layer is deposited in those areas of the composite layer not coated with the blocking agent. Any sort of blocking agent is useful in the patch coating of the present invention. Blocking agents useful in the present invention include, but are not limited to, silicone primer (i.e., MED6-161). Other silicone primers and blocking agents are useful in the invention.

The conducting polymer layer can be coated on the polymer substrate with the biocompatible conductive material. It can be coated in either a patch coating form described above or can be coated completely and then the correct shape can be die cut from it. Two such biocompatible polymer substrates with conductive materials coated with conducting polymer layers that have been die cut to the correct shape, can be placed on two sides of the insulating polymer layer and the assembly aligned to stagger the conducting polymer layers as shown in FIGS. 56B, C, D and E. The final product is then assembled by bonding together the layers via hot pressing or lamination to form the assembly. The silicone coating and patch coating are useful for preventing or reducing delamination of the conductive polymer layer from the composite layer. The silicone coating and patch coating are also useful for preventing or reducing the formation of cracks and bubbles in the conductive polymer layer.

### V. Device Embodiments

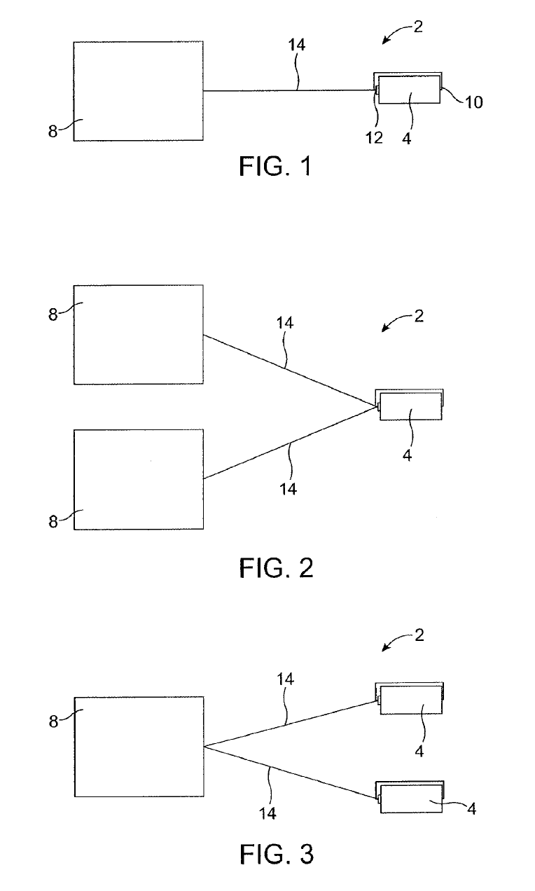

FIG. 1 illustrates an airway implant system 2 that has a power source 4, a connecting element, such as a wire lead 14, and an actuator element, such as an electroactive polymer element 8. Suitable power sources 4 are a power cell, a battery, a capacitor, a substantially infinite bus (e.g., a wall outlet leading to a power generator), a generator (e.g., a portable generator, a solar generator, an internal combustion generator), or combinations thereof. The power source 4 typically has a power output of from about 1 mA to about 5 A, for example about 500 mA.

Instead of or in addition to wire lead 14, the connecting element may be an inductive energy transfer system, a conductive energy transfer system, a chemical energy transfer system, an acoustic or otherwise vibratory energy transfer system, a nerve or nerve pathway, other biological tissue, or combinations thereof. The connecting element is made from one or more conductive materials, such as copper. The connecting element is completely or partially insulated and/or protected by an insulator, for example polytetrafluoroethylene (PTFE). The insulator can be biocompatible. The power source 4 is typically in electrical communication with the actuator element 8 through the connecting element. The connecting element is attached to an anode 10 and a cathode 12 on the power source 4. The connecting elements can be made from one or more sub-elements.

The actuator element 8 is preferably made from an electroactive polymer element, as described above.

FIG. 2 illustrates that the actuator element 8 can have multiple elements 8 and connecting elements 14 that all connect to a single power source 4.

FIG. 3 illustrates an airway implant system 2 with multiple power sources 4 and connecting elements 14 that all connect to a single actuator element 8. The airway implant system 2 can have any number and combination of actuator elements 8 connected to power sources 4.

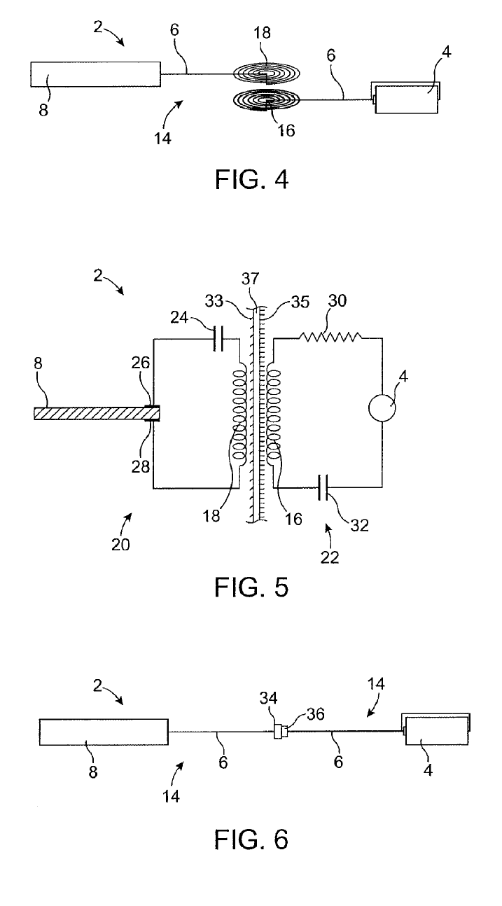

FIG. 4 illustrates an embodiment with the connecting element having a first energy transfer element, for example a first receiver, and a second energy transfer element, for example a second receiver such as a second inductor 16. In this embodiment, the first receiver is a first inductor 18. The first inductor 18 is typically positioned close enough to the second inductor 16 to enable sufficient inductive electricity transfer between the second and first inductors 16 and 18 to energize the actuator element 8. The connecting element 14 has multiple connecting elements 6.

FIG. 5 illustrates that the airway implant device of the present invention can have an implanted portion 20 and a non-implanted portion 22. In this embodiment, the implanted portion 20 is a closed circuit with the first inductor 18 in series with a first capacitor 24 and the actuator element 8. The actuator element 8 is attached to the closed circuit of the implanted portion 20 by a first contact 26 and a second contact 28. In some embodiments, the implanted portion has a resistor (not shown). The non-implanted portion 22 is a closed circuit. The non-implanted portion 22 has a second inductor 16 that is in series with a resistor 30, the power source 4, and a second capacitor 32. The capacitors, resistors, and, in-part, the inductors are representative of the electrical characteristics of the wire of the circuit and not necessarily representative of specific elements. The implanted portion 20 is within tissue and has a tissue surface 33 nearby. The non-implanted portion is in insulation material 35. An air interface 37 is between the tissue surface 33 and the insulation material 35.

FIG. 6 illustrates an embodiment in which the first energy transfer element of the connecting element 14 is a first conductor 34. The second energy transfer element of the connecting element 14 is a second conductor 36. The first conductor 34 is configured to plug into, receive, or otherwise make secure electrical conductive contact with the second conductor 36. The first conductor 34 and/or second conductor 36 are plugs, sockets, conductive dental fillings, tooth caps, fake teeth, or any combination thereof.

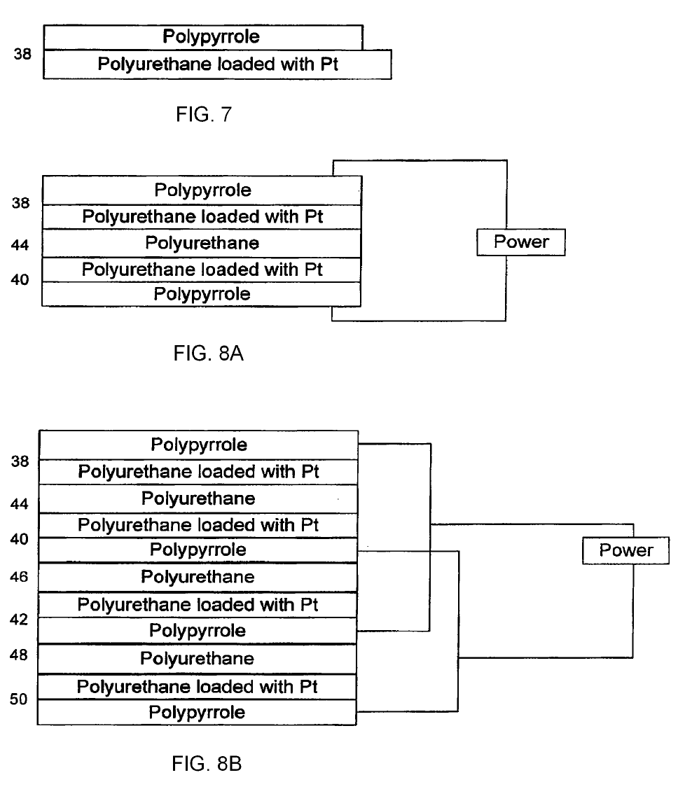

FIG. 7 illustrates an embodiment in which the actuator element 8 is a single-layered device having a first EAP layer 38. As shown in FIG. 7, the single layer EAP includes a composite layer of polyurethane and Pt, with a polypyrrole conductive polymer layer disposed on one of the opposing surfaces of the composite layer.

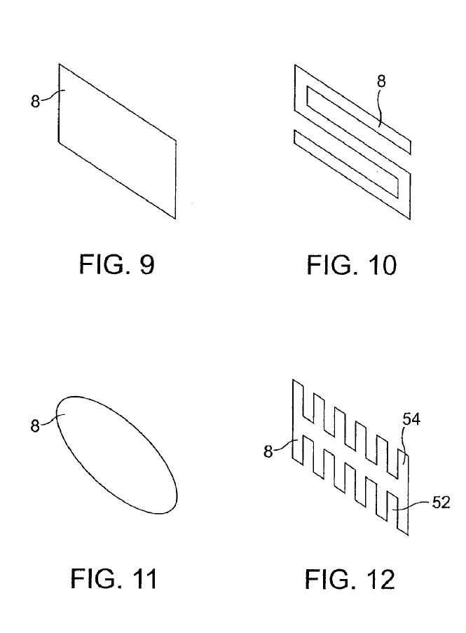

FIGS. 8A and 8B illustrate additional embodiments in which the actuator element 8 has multiple layers. FIG. 8A illustrates a bimorph structure having a first EAP layer 38 separated from a second EAP layer 40 by a first insulation layer 44. FIG. 8B illustrates a multilayer structure having the bimorph structure along with a second insulation layer 46 separating the second EAP layer from the third EAP layer 42. A third insulation layer 48 separates the third EAP layer from the fourth EAP layer 50. Insulation material is preferably a polymeric material that electrically isolates each layer. The insulation can be, for example, acrylic polymers, polyimide, polypropylene, polyethylene, silicones, nylons, polyesters, polyurethanes, or combinations thereof. Each EAP layer, 38, 40, 42 and 50 can be connected to a lead wire (not shown). All anodes and all cathodes are connected to the power source 4.

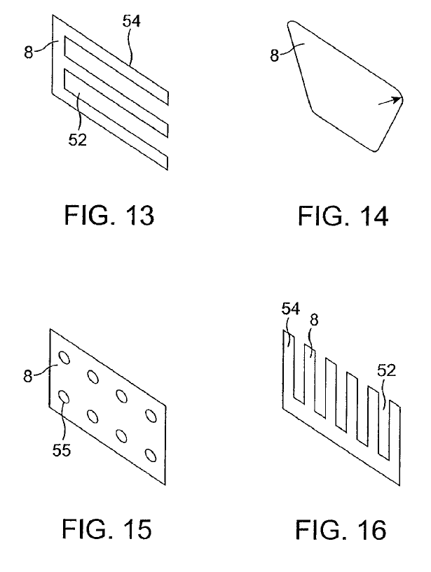

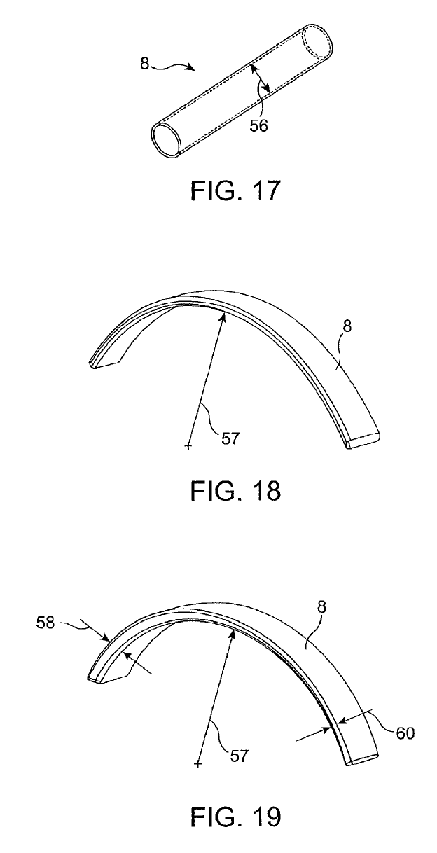

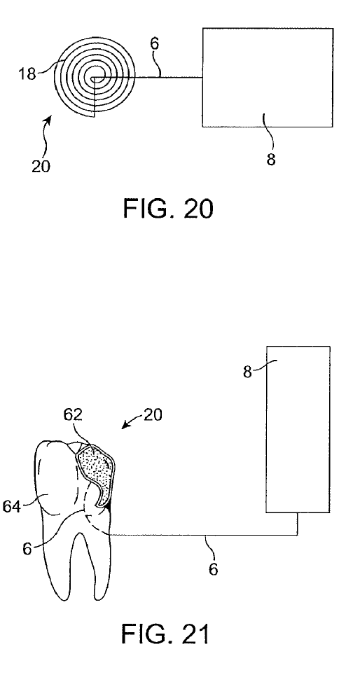

FIGS. 9-19 illustrate different suitable shapes for the actuator element 8. FIG. 9 illustrates an actuator element 8 with a substantially flat rectangular configuration. The actuator element 8 can have a width from about 2 mm to about 5 cm, for example about 1 cm. FIG. 10 illustrates an actuator element 8 with an “S” or zig-zag shape. FIG. 11 illustrates the actuator element 8 with an oval shape. FIG. 12 illustrates an actuator element 8 with a substantially flat rectangular shape with slots 52 cut perpendicular to the longitudinal axis of the actuator element 8. The slots 52 originate near the longitudinal axis of the actuator element 8. The actuator element 8 has legs 54 extending away from the longitudinal axis. FIG. 13 illustrates an actuator element 8 with slots 52 and legs 54 parallel with the longitudinal axis. FIG. 14 illustrates an actuator element be configured as a quadrilateral, such as a trapezoid. The actuator element 8 has chamfered corners, as shown by radius. FIG. 15 illustrates an actuator element 8 with apertures 55, holes, perforations, or combinations thereof. FIG. 16 illustrates a actuator element 8 with slots 52 and legs 54 extending from a side of the actuator element 8 parallel with the longitudinal axis. FIG. 17 illustrates an actuator element 8 with a hollow cylinder, tube, or rod. The actuator element has an inner diameter 56. FIG. 18 illustrates an arched actuator element 8. The arch has a radius of curvature 57 from about 1 cm to about 10 cm, for example about 4 cm. The actuator element 8 has a uniform thickness. FIG. 19 illustrates an arched actuator element 8. The actuator element 8 can have a varying thickness. A first thickness 58 is equal or greater than a second thickness 60.

FIG. 20 illustrates an embodiment of the implanted portion of an airway implant with a coil-type inductor 18 connected by a wire lead 6 to the actuator element 8. In another embodiment, as illustrated in FIG. 21 the implanted portion has a conductive dental filling 62 in a tooth 64. The dental filling 62 is previously implanted for reasons related or unrelated to using of the airway implant system. The dental filling 62 is electrically connected to the wire lead 6. For example, a portion of the wire lead 6 is implanted in the tooth 64, as shown by phantom line. The wire lead 6 is connected to the actuator element 8.

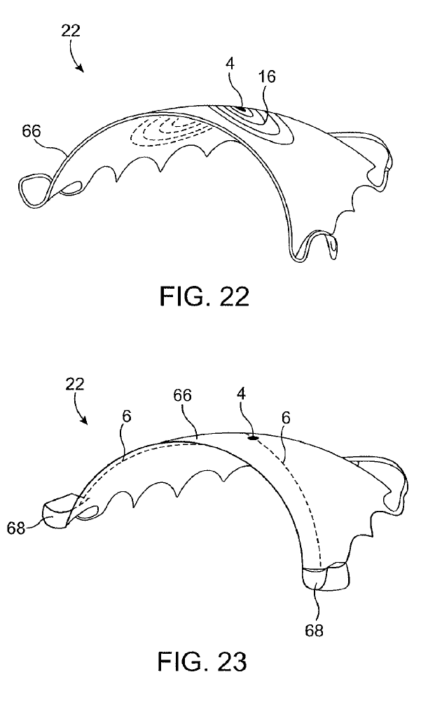

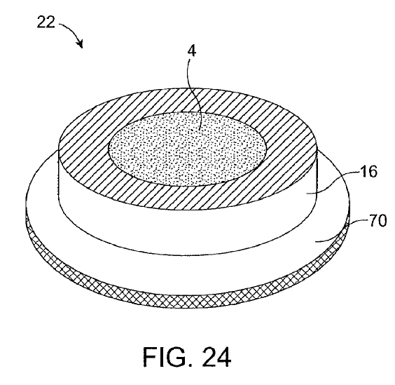

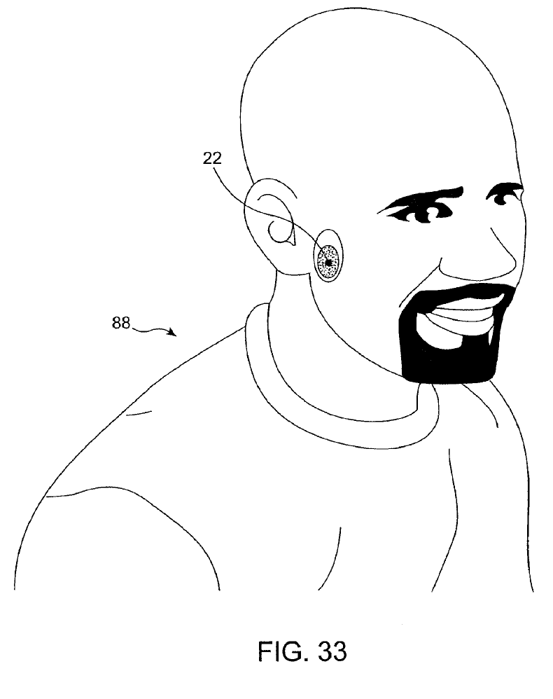

FIG. 22 illustrates an embodiment of the non-implanted portion 22 with a mouthpiece, such as a mouthpiece 66. The mouthpiece 66 is preferably custom configured to fit to the patient's mouth roof, or another part of the patient's mouth. The second receiver, such as second inductor 16, is integral with, or attached to, the mouthpiece 66. The second inductor 16 is located in the mouthpiece 66 so that during use the second inductor 16 is proximal with the first inductor 18. The power source 4, such as a cell, is integral with, or attached to, the mouthpiece 66. The power source 4 is in electrical communication with the second inductor 16. In some embodiments, the mouthpiece 66 has a pulse-width-modulation circuit. FIG. 23 illustrates that the mouthpiece 66 has one or more tooth sockets 68. The tooth sockets 68 are preferably configured to receive teeth that have dental fillings. The tooth sockets 68 are electrically conductive in areas where they align with dental fillings when in use. The power source 4 is connected with the tooth sockets 68 via the wire leads 6. In the embodiment of FIG. 24, the non-implantable portion 22 has the second inductor 16 attached to a removably attachable patch 70. The patch 70 is attached to the power source 4. The power source 4 is in contact with the second inductor 16. This embodiment can be, for example, located on the cheeks as shown on FIG. 33 or any other suitable location.

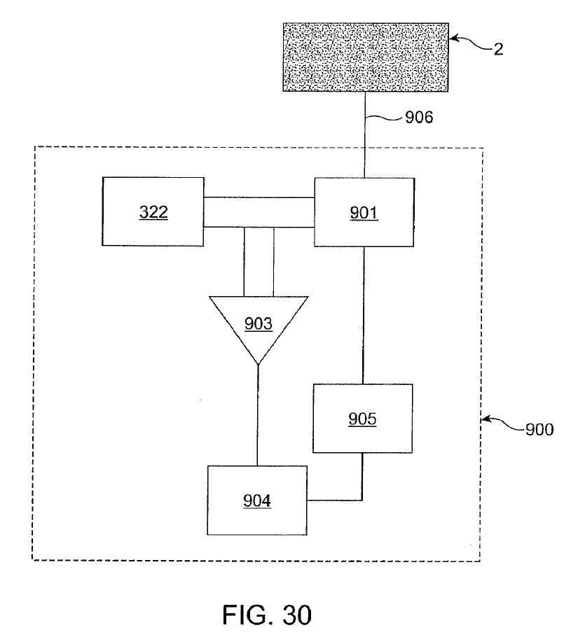

Preferably, the airway implant device 2 discussed herein is used in combination with an inductive coupling system 900 such as depicted in FIG. 30. FIG. 30 depicts an inductive coupling system that is suitable for controlling the airway implant device 2 which includes a connecting element 906 (which connects the electrical contacts (not shown) to the rest of the electrical system), a connector 901, a energy source 322, a sensor 903, a timer 904, and a controller 905. The connector 901, energy source 322, sensor 903, a timer 904, and controller 905 are located in a housing disposed in a region outside or inside the body. The sensor can be used to sense when the EAP is energized.

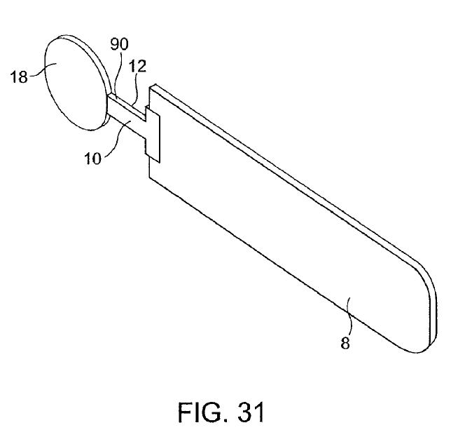

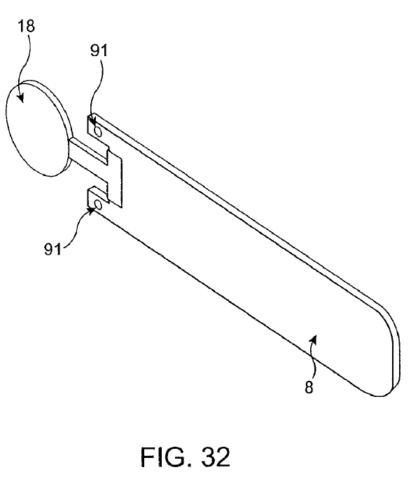

Two preferred embodiments of the airway implant device are shown in FIGS. 31 and 32. The device in FIG. 31 includes the actuator element 8 connected to an anode 10 and cathode 12 and to the induction coil 18. The device also includes a controller 90, such as a microprocessor. The circuitry within the controller is not shown. The controller 90 picks up AC signals from the induction coil 18 and converts it to DC current. The controller 90 can also include a time delay circuit and/or a sensor. FIG. 32 shows an embodiment with anchors 91 located on the actuator element 8. The implant can be anchored in a suitable location with the use of these anchors and sutures and/or surgical glue.

Another preferred embodiment of the airway implant device is shown in FIG. 52. The device 5250 in FIG. 52 shows a silicone rubber coating 5251 coating a portion of the electroactive polymer layer 5211 and attached to acrylic hub 5212. In the absence of the silicon coating 5251, the electroactive polymer layer 5211 extends to the acrylic hub 5212, see device 5210.

FIG. 53 shows a device 5310 of the invention with the conductive polymer layer 5330 patch coating the composite layer 5320.

FIG. 56 shows several embodiments of the electroactive polymer element of the device of the present invention. FIG. 56A shows the composite layer 5610 completely coated on both opposing surfaces by the conductive polymer layer 5620. FIG. 56B shows the composite layer 5610 completely coated on one opposing surfaces by the conductive polymer layer 5620 and partially coated on the other opposing surface by the several patches 5630 of conductive polymer layer. FIGS. 56B, 56C, 56D and 56E show several embodiments of the composite layer 5610 coated on both opposing surfaces by patches 5630 of the conductive polymer layer, where each opposing surface of the composite layer 5610 has a different number of patches 5630 of the conductive polymer layer, or a different spacing between the patches 5630 of conductive polymer layer.

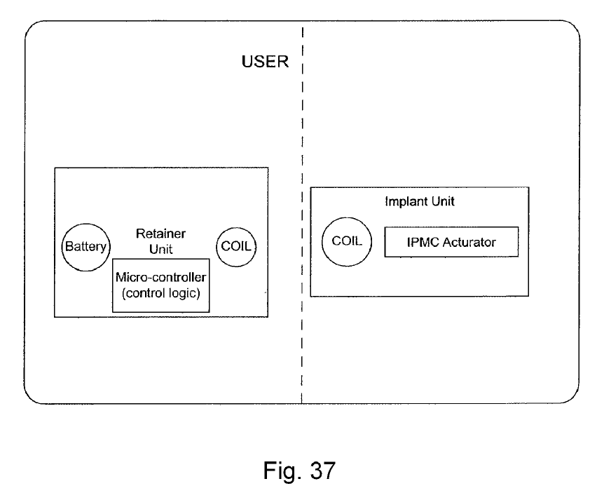

FIG. 37 depicts an embodiment of the invention. The airway implant device can be of two units—an implant unit and a mouthpiece unit. The implant unit is implanted in a patient and includes an IPMC actuator and a coil. The mouthpiece unit is typically not implanted in the patient and can be worn by the patient prior to going to bed. This unit includes a coil, a battery, and a microcontroller.

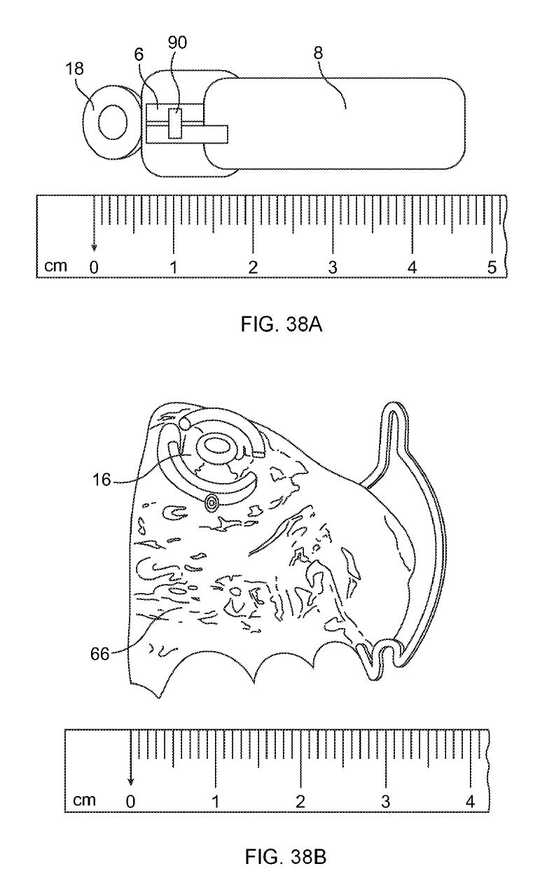

FIG. 38 depicts yet another embodiment of the invention. FIG. 38A is the implant unit, preferably for implantation proximal to or in an airway wall. The implant unit includes an actuator element 8, an inductor 18 in the form of a coil, a controller 90, and connecting elements 6. FIG. 38B depicts the removable mouthpiece with an inductor 16 and a mouthpiece 66.

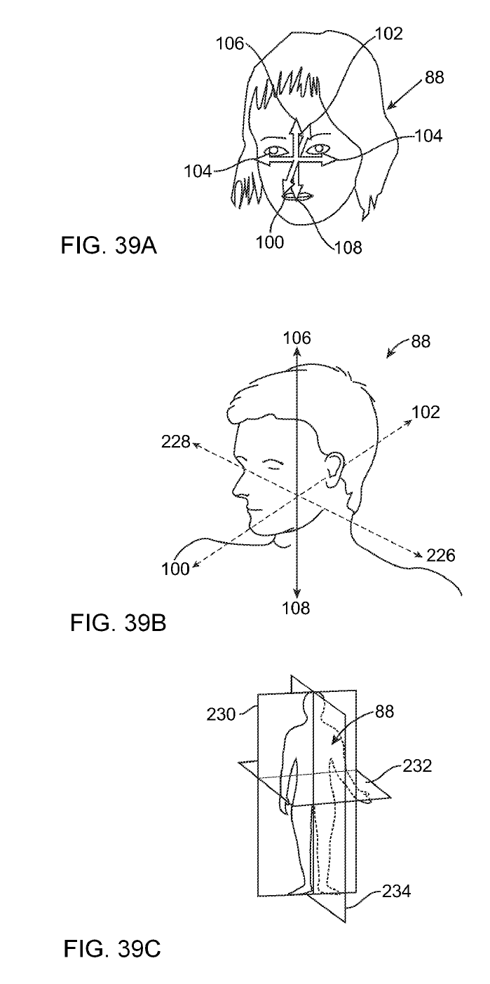

FIGS. 39A, 39B, and 39C illustrate terms used in describing the anatomy of a patient 88 and orientation attributes of the invention. Anterior 100 refers to a part of the body or invention toward the front of the body or invention, or in front of another part of the body or invention. Posterior 102 refers to a part of the invention or body toward the back of the invention or body, or behind another part of the invention or body. Lateral 104 refers to a part of the invention or body to the side of the invention or body, or away from the middle of the invention or body or the middle of the invention or body. Superior 106 refers to a part of the invention or body toward the top of the invention or body. Inferior 108 refers to a part of the invention or body toward the bottom of the invention or body. FIG. 39B illustrates the left 226 and the right 228 sides of a patient anatomy. Various planes of view are illustrated in FIG. 39C, including a coronal plane 230, a transverse plane 232, and a sagittal plane 230.

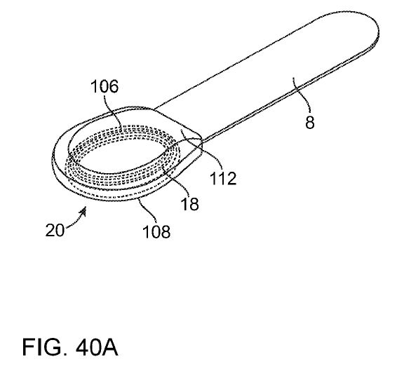

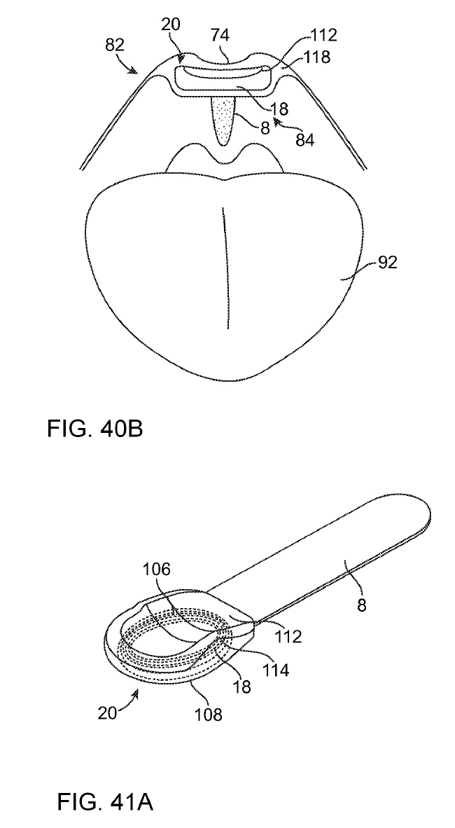

FIG. 40A illustrates one embodiment of the airway implant device having a actuator element 8, a first inductor 18, and a housing 112 made from an acrylic and cast with substantially smooth rounded superior and anterior sides. In this embodiment, the actuator element 8 anterior end terminates at about the posterior end of the acrylic housing 112. FIG. 40B illustrates the implant device of FIG. 40A viewed from the anterior side of the implant device, looking toward the posterior end, wherein the implant device is implanted in the palate 116. In the embodiment shown in FIG. 40B, the implant device is implanted such that the housing 112 is in the periosteum 118 inferior to the ridge of the hard palate 74, and the actuator element 8 extends into the soft palate 84.

A preferred embodiment of the device of the present invention can be an implanted portion 20 having an implantable actuator element 8, a housing 112, a first inductor 18, and connecting elements 14 connecting the actuator element 8 to the first inductor 18 within the housing 112; and a non-implanted portion 22 having a power source 4 and a second inductor 16 capable of transferring energy to the first inductor 18, wherein the energy of the first inductor 18 energizes the actuator element 8 wherein the actuator element 8 can be an electroactive polymer element. In a preferred embodiment, the actuator element 8 of the device is implanted in the soft palate 84. The housing 112 of the preferred embodiment is implanted inferior to the hard palate 74. In a preferred embodiment of the device, the housing 112 can be at least one of acrylic, polytetrafluoroethylene (PTFE), polymethylmethacrylate (PMMA), Acrylonitrile Butadiene Styrene (ABS), polyurethane, polycarbonate, cellulose acetate, nylon, and a thermoplastic or thermosetting material.

In a preferred embodiment, the non-implanted portion 22 is in the form of a mouthpiece 66. In a preferred embodiment, the non-implanted portion can be a non-implantable wearable element. In some embodiments, the superior side of the housing 112 comports to the shape of a hard palate 74. In some embodiments, the housing 112 is cast from an impression of a hard palate 74. In still other embodiments, the housing 112 is concave on its superior side. In some embodiments, the housing 112 is convex on its superior side. In some embodiments, the housing 112 can be bumps 114 on its superior side lateral to a central axis extending from the housing's 112 anterior to its posterior end. In some embodiments, the housing 112 configuration has a substantially smooth rounded superior side. Other configurations for the housing 112 may be contemplated by one having skill in the art without departing from the invention.

In some embodiments, the actuator element 8 is at least partially within the housing 112. In other embodiments, the actuator element 8 is outside the housing 112. The housing 112 is capable of housing and protecting the first inductor 18 and connecting elements 14 between the first inductor 18 and the actuator element 8. In some embodiments, the housing 112 has a roughened surface to increase friction on the housing 112. In some embodiments, the roughened surface is created during casting of the housing 112. In some embodiments, the roughened surface induces fibrosis.

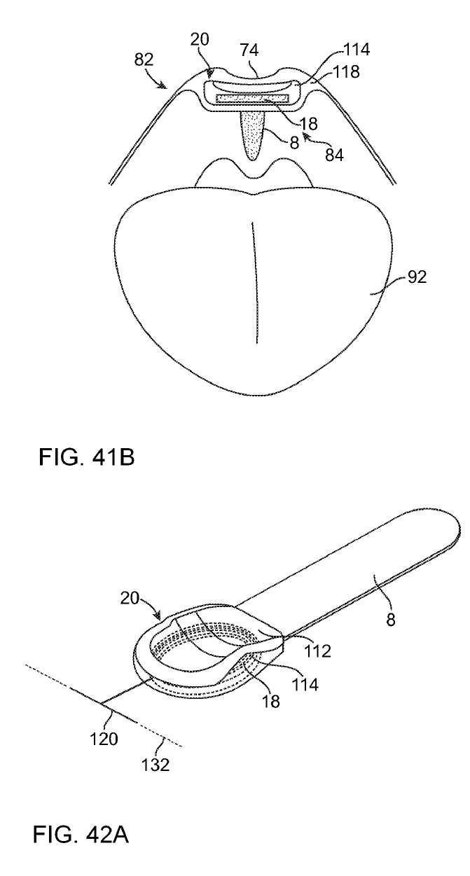

FIG. 41A illustrates an embodiment of the airway implant device that has a actuator element 8, a first inductor 18, and a housing 112 with a smooth rounded inferior side, and at least two bumps 114 on its superior side which, when implanted, comport with the lateral sides of the ridge of the hard palate 74, as shown in FIG. 41B. This configuration reduces rocking of the implant device on the ridge of the hard palate 74 when implanted. In this embodiment, the actuator element 8 anterior end terminates at about the posterior end of the acrylic housing 112. FIG. 41B illustrates the airway implant device of FIG. 41A, viewed from the anterior side of the implant, looking toward the posterior end, wherein the implant device is implanted in the palate 116. In the embodiment shown in FIG. 41B, the implant device is implanted such that the housing 112 is in the periosteum 118 inferior to the ridge of the hard palate 74, and the actuator element 8 extends into the soft palate 84.

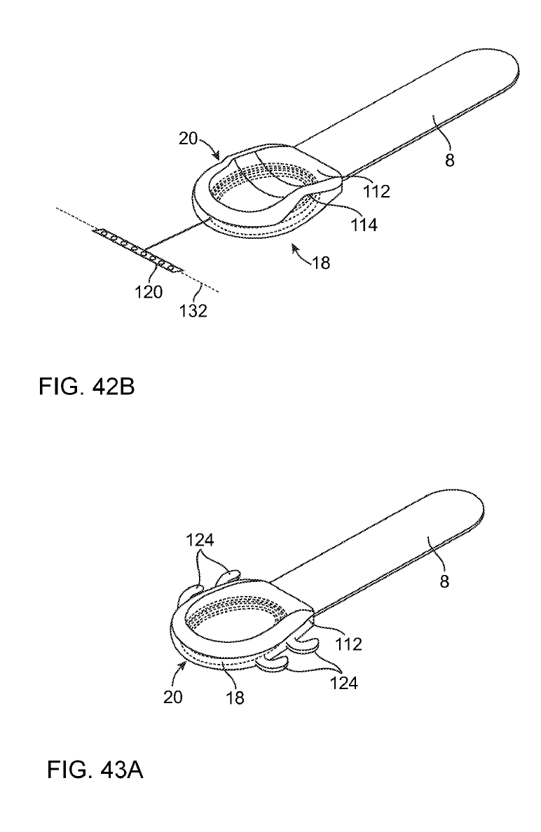

FIG. 42A illustrates an embodiment of the airway implant device having an attachment element 120 at the anterior end of the implant. In this embodiment, the attachment element 120 is T-shaped, however, other configurations and geometries of the attachment element 120 are contemplated in other embodiments, including triangular, circular, L-shaped, Z-shaped, and any geometry within the contemplation of one skilled in the art that would allow attachment of the attachment element to tissue at the anterior end of the implant to fix the position of the implant within the implant cavity.

In some embodiments of the airway implant device having attachment elements 120, the attachment element 120 is a bioabsorbable material. Examples of bioabsorbable materials include, but are not limited to, polylactic acid, polyglycolic acid, poly(dioxanone), Poly(lactide-co-glycolide), polyhydroxybutyrate, polyester, poly(amino acid), poly(trimethylene carbonate) copolymer, poly (ε-caprolactone) homopolymer, poly (ε-caprolactone) copolymer, polyanhydride, polyorthoester, polyphosphazene, and any bioabsorbable polymer.

In another embodiment, the airway implant device can be an attachment element 120, as shown in FIG. 42B wherein the perforated attachment element 120 can be at least one hole 122. The hole provides a means for a suture or other attaching device to affix the device to tissue and secure the implant device position. In the case where a suture 132 is used, the suture may or may not be the same suture used by a practitioner to close the original incision made to create a cavity for the implant. The attaching device can be at least one of a suture, clip, staple, tack, and adhesive.

In some embodiments, the implant may be secured in place, with or without use of an attachment element 120, using an adhesive suitable for tissue, such as cyanoacrylates, and including, but not limited to, 2-octylcyanoacrylate, and N-butyl-2-cyanoacrylate.

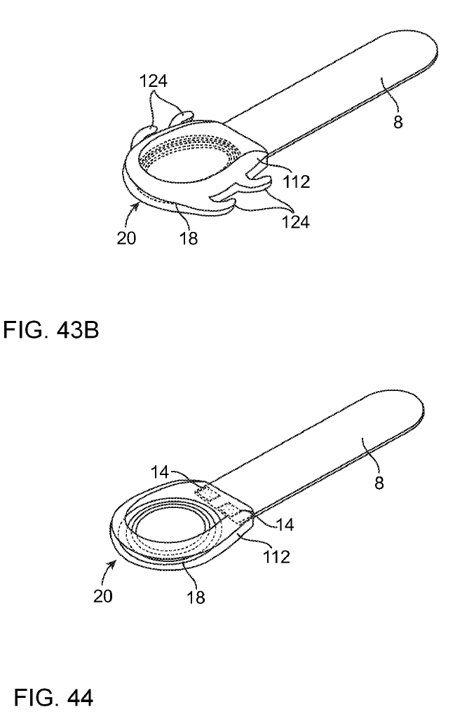

FIGS. 43A and 43B illustrate an embodiment of the airway implant device wherein the housing 112 has at least one anchor 124. In FIGS. 43A and 43B, the device has four saw-blade like directional anchors 124. The anchors 124 may or may not be made of made of the same materials as the housing 112. Such materials include at least one of acrylic, polytetrafluoroethylene (PTFE), polymethylmethacrylate (PMMA), Acrylonitrile Butadiene Styrene (ABS), polyurethane, polycarbonate, cellulose acetate, nylon, and a thermoplastic material. In some embodiments, the device has at least one anchor 124. In some embodiments, the anchor 124 is configured to allow delivery and removal of the implant device with minimal tissue damage. In some embodiments, the anchor 124 is curved. In some embodiments the superior side(s) of the anchor(s) 124 comport with the hard palate 74 surface, FIG. 43A. In other embodiments, the superior side(s) of the anchor(s) 124 conform to the configuration of the housing 112, options for which are as described elsewhere in this disclosure, FIG. 43B.

FIG. 44 illustrates a preferred embodiment of the airway implant device wherein the implanted portion 20 can be power connecting elements 14 having a first contact 26 and a second contact 28. In this embodiment, the first contact 26 and second contact 28 have opposing electrical charges, and the housing 112 encases the contacts. In the embodiment shown, the first contact 26 faces in the inferior direction, while the second contact 28 faces in the superior direction. In other embodiments, the first contact 26 faces in the superior direction while the second contact 28 faces in the inferior direction. In some embodiments, the connecting element 14 can be a non-corrosive conductive material. In some embodiments, the connecting element 14 can be platinum, gold, silver, stainless steel, or conductive carbon. In some embodiments, the connecting element 14 can be stainless steel or copper plated with gold, platinum, or silver. In some embodiments, the actuator element 8 stiffens in one direction when a charge is applied to the connecting element 14. In some embodiments, the actuator element 8 deflects when a charge is applied to the connecting element 14.

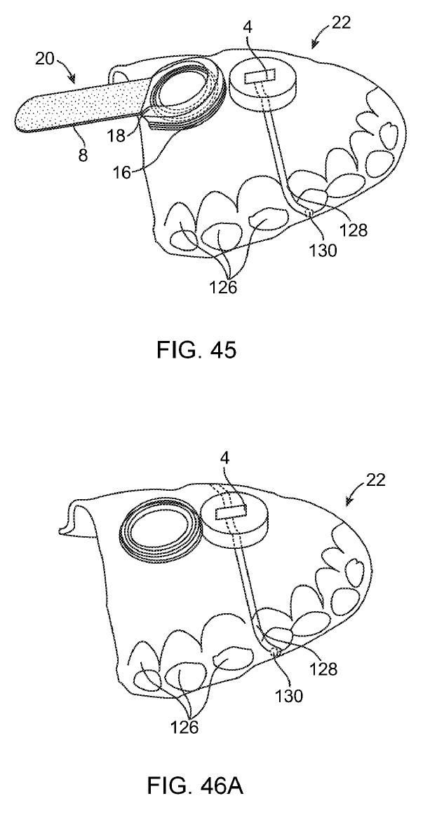

FIG. 45 illustrates an embodiment of the airway implant system wherein the device can be a non-implanted portion 22 in the form of, and made from similar material as a dental mouthpiece 66. The mouthpiece 66 depicted in FIG. 45 has teeth impressions 126 corresponding to a patient's approximate or exact dentition. Example dental mouthpiece materials include acrylate, polymethylmethacrylate (PMMA), polycarbonate, and nylon. In the embodiment shown in FIG. 45, the non-implanted portion can be a power source 4 that is rechargeable, a second inductor 16 connected to the power source 4, and ball clamps 128 having two exposed portions 130, the ball clamps 128 connected to the rechargeable power source 4, whereby the exposed portions 130 can recharge the power source 4. The exposed portions 130 are at least partially not covered by mouthpiece material, and are thereby exposed. In the embodiment shown in FIG. 45, the non-implanted portion second inductor 16 transfers energy it receives from the power source 4 to the first inductor 18 of the implanted portion 20, wherein the first inductor 18 energizes the actuator element 8.

In some embodiments, the non-implanted portion 22 does not include ball clamps 128 for recharging the power source 4. In some embodiments, the power source 4 is a rechargeable battery. In some embodiments, the power source 4 is one of a lithium-ion battery, lithium-ion polymer battery, a silver-iodide battery, lead acid battery, a high energy density, or a combination thereof. In some embodiments, the power source 4 is removable from the non-implanted portion 22. In some embodiments, the power source 4 is replaceable. In some embodiments, the power source is designed to be replaced or recharged per a specified time interval. In some embodiments, replacing or recharging the power source 4 is necessary no more frequently than once per year. In other embodiments, replacing or recharging the power source 4 is necessary no more frequently than once every six months. In yet other embodiments, replacing or recharging the power source 4 is necessary no more frequently than once or every three months. In yet another embodiment, daily replacing or recharging of the power source is required.

In some embodiments, the power source 4 and second inductor 16 are sealed within the non-implanted portion and the sealing is liquidproof.

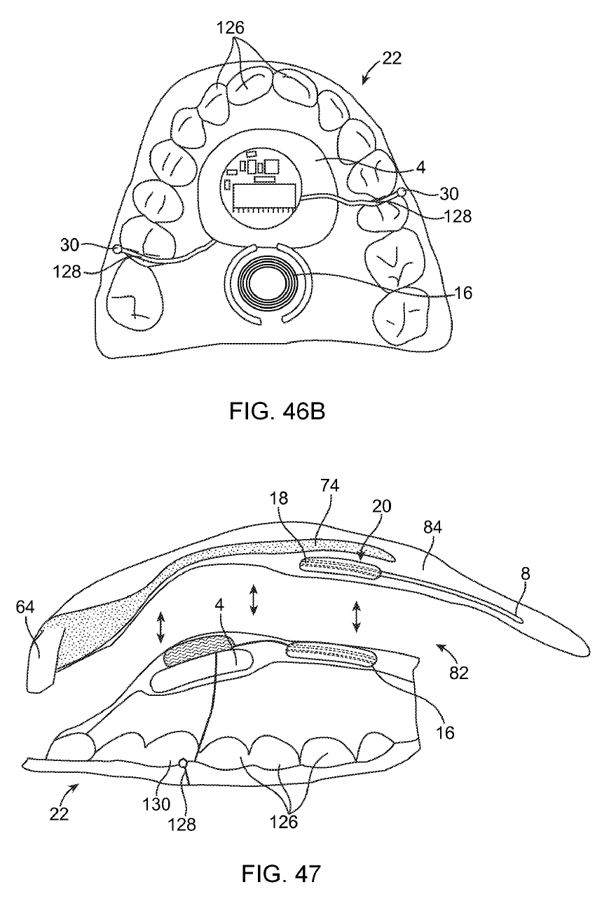

FIGS. 46A, and 46B illustrate different views of an embodiment of the airway implant device non-implanted portion 22 in the form of a mouthpiece 66. In the embodiment depicted, the non-implanted portion 22 can be a second inductor 16, a power source 4, and at least one ball clasp 128 for recharging the power source 4.

FIG. 47 illustrates an embodiment of the airway implant device implanted in the palate 116. In this embodiment, the housing 112 is implanted inferior to the hard palate 74, whereas the actuator element 8 extends posterior to the housing 112 into the soft palate 84. The non-implanted portion 22 in this embodiment can be a mouthpiece 66, a power source 4, a second inductor 16, and ball clamps 128 for recharging the power source 4. Other embodiments can have none, or some, or all of these elements (the mouthpiece 66, power source 4, second inductor 16, and ball clamps 128), and instead open the airway by means described elsewhere in this specification. In the embodiment depicted in FIG. 47, when the implanted portion 20 of the airway implant device is implanted such that the housing 112 is inferior to the hard palate 74, and when a patient places the mouthpiece 66 in his mouth 82, the mouthpiece 66 having a chargeable second inductor 16 that is positioned within the mouthpiece 66 to align inferior to the implanted first inductor 18, the second inductor 16 transfers energy to the first inductor 18 and the first inductor 18 energizes the actuator element 8. In this embodiment, the actuator element 8 can be an electroactive polymer (EAP) element, which, when energized by the first inductor 18, opens the airway in which the device is implanted.

The implants described herein are preferably implanted with a deployment tool. Typically, the implantation involves an incision, surgical cavitation, and/or affixing the implant.

### VI. Device for Stabilizing the Tongue

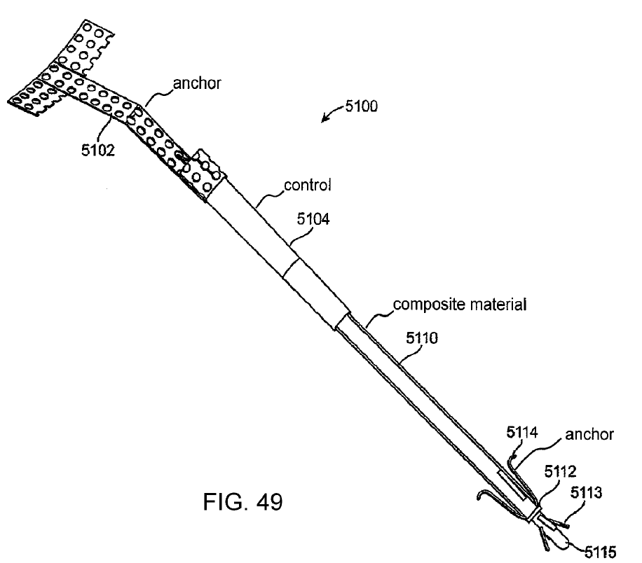

In some embodiments, the present invention provides an implant for stabilizing the tongue. The implant can have a first anchoring portion for securing a first end of the implant with the mandibula, such as 5112 in FIG. 49. The implant further has a control portion connected with the anchoring portion, configured for selectively activating the implant. A flexible portion can be connected at its proximal end with the control portion, the flexible portion having three-dimensional flexibility in a non-energized state and the flexible portion having a lesser three-dimensional flexibility in a energized state, the flexible portion being selectively switchable between the non-energized and energized states by the control portion (see 5108 of FIG. 49). The flexible portion can be an electroactive polymer element having a composite layer having a polymer substrate and a biocompatible conductive material, wherein the composite layer also includes opposing surfaces and a conductive polymer layer disposed on at least one of the opposing surfaces of the composite layer (see 8 of FIG. 48 and FIGS. 7 and 8). The implant can further include a second anchoring portion connected with the flexible portion, the second anchoring portion being configured for connecting the implant with the base of the tongue, such as bracket 5102 in FIG. 49.

In some embodiments, the present invention provides a method of controlling an opening of an air passageway, including: implanting an airway implant device proximal to an air passageway, in a wall of an air passageway or in both, the device having an electroactive polymer element having: a composite layer having a polymer substrate and a biocompatible conductive material, wherein the composite layer also includes opposing surfaces; and a conductive polymer layer disposed on at least one of the opposing surfaces of the composite layer, wherein the implant device is adapted and configured to modulate an opening of an air passageway; and energizing the electroactive polymer element for a fixed period of time, such that the electroactive polymer element adopts an energized state and maintains the energized state after the fixed period of time has passed, thereby completely or partially opening the air passageway.

In another embodiment, the method also includes de-energizing the electroactive polymer element to a non-energized state. In other embodiments, the implantation of the airway implant device is in a soft palate, a lateral pharyngeal wall, a tongue or a combination thereof. In still other embodiments, the airway implant device is controlled by an inductive coupling mechanism.

In a further embodiment, the present invention provides a method of treating a disease using an airway implant device, having: implanting an airway implant device proximal to an air passageway or in a wall of an air passageway or in both, the device having an electroactive polymer element having: a composite layer having a polymer substrate and a biocompatible conductive material, wherein the composite layer also includes opposing surfaces; and a conductive polymer layer disposed on at least one of the opposing surfaces of the composite layer, wherein the implant device is adapted and configured to modulate an opening of an air passageway; and energizing the electroactive polymer element for a fixed period of time, such that the electroactive polymer element adopts an energized state and maintains the energized state after the fixed period of time has passed, thereby treating the disease.

In some embodiments, the disease is obstructive sleep apnea and/or snoring. In other embodiments, the airway implant device is controlled by an inductive coupling mechanism. In still other embodiments, the airway implant device is implanted in a soft palate, and the energizing of the electroactive polymer element supports the soft palate. In yet other embodiments, the airway implant device is implanted in a lateral pharyngeal wall, and the energizing of the electroactive polymer element prevents the lateral pharyngeal wall from collapsing. In still yet other embodiments, the airway implant device is implanted in a tongue, and the energizing of the electroactive polymer element prevents the tongue from collapsing.

In another embodiment, the present invention provides an implant for stabilizing the tongue, having: a first anchoring portion for securing a first end of the implant with the mandibula; a control portion connected with the anchoring portion, configured for selectively activating the implant; a flexible portion connected at its proximal end with the control portion, the flexible portion having three-dimensional flexibility in a non-energized state and the flexible portion having a lesser three-dimensional flexibility in a energized state, the flexible portion being selectively switchable between the non-energized and energized states by the control portion, wherein the flexible portion can be an electroactive polymer element having: a composite layer having a polymer substrate and a biocompatible conductive material, wherein the composite layer also includes opposing surfaces; and a conductive polymer layer disposed on at least one of the opposing surfaces of the composite layer; and a second anchoring portion connected with the flexible portion, the second anchoring portion being configured for connecting the implant with the base of the tongue.

In some embodiments, the first anchoring portion includes an anchoring bracket. In other embodiments, the control portion is powered by a non-implanted inductively coupled power source. In some other embodiments, the flexible portion is coated with a hyaluronic acid coating. In still other embodiments, the second anchoring portion can be a first disc connected with the distal end of the flexible portion and a second disc connectible with the base of the tongue. In yet other embodiments, the first and second discs include suture holes disposed around their circumferences. In still yet other embodiments, the first and second discs also include polyester rods having holes extending from the flat surfaces of the discs.

In a further embodiment, the second anchoring portion can be a first proximally extending anchor portion and a second distally extending anchor portion. In other embodiments, the device also includes a coating to prevent tissue in-growth. In some other embodiments, the device also includes a coating to promote tissue growth.

In another embodiment, the present invention provides a method of treating a disease using an airway implant device, having: implanting in a subject's tongue a device having a first anchoring portion for securing a first end of the implant with the mandibula; a control portion connected with the anchoring portion, configured for selectively activating the implant; a flexible portion connected at its proximal end with the control portion, the flexible portion having three-dimensional flexibility in a non-energized state and the flexible portion having a lesser three-dimensional flexibility in a energized state, the flexible portion being selectively switchable between the non-energized and energized states by the control portion, wherein the flexible portion can be an electroactive polymer element having: a composite layer having a polymer substrate and a biocompatible conductive material, wherein the composite layer also includes opposing surfaces; and a conductive polymer layer disposed on at least one of the opposing surfaces of the composite layer; a second anchoring portion connected with the distal end of the flexible portion, the second anchoring portion being configured for connecting the implant with the base of the tongue; and supporting the subject's tongue by selectively activating the control portion of the implant.

In some embodiments, the disease is a sleep disorder. In other embodiments, the sleep disorder is an obstructive sleep apnea or snoring.

In a further embodiment, the present invention provides a method of treating a disease using an airway implant device having: implanting in a subject's tongue a device having a first anchoring portion for securing a first end of the implant with the mandibula; a control portion connected with the anchoring portion, configured for selectively activating the implant; a flexible portion connected at its proximal end with the control portion, the flexible portion having three-dimensional flexibility in a non-energized state and the flexible portion having a lesser three-dimensional flexibility in a energized state, the flexible portion being selectively switchable between the non-energized and energized states by the control portion, wherein the flexible portion can be an electroactive polymer element having: a composite layer having a polymer substrate and a biocompatible conductive material, wherein the composite layer also includes opposing surfaces; and a conductive polymer layer disposed on at least one of the opposing surfaces of the composite layer; a second anchoring portion connected with the distal end of the flexible portion, the second anchoring portion being configured for connecting the implant with the base of the tongue; an inductive powering mechanism coupled with the control portion and configured to maintain the flexible portion in either of the non-energized and energized states, the device being adapted and configured to support the tongue upon being energized; and supporting the subject's tongue by selectively activating the control portion of the implant using the inductive powering mechanism.

One aspect of the invention is an airway implant device with a connecting element. Preferably the connecting element is used to anchor and/or support the airway implant device, in particular, the electroactive polymer element to a rigid structure, such as a bony structure. The invention also includes methods of treating a disease using an airway implant device by implanting in a subject the airway implant device having an electroactive polymer element and a connecting element, the implanting step including fastening the electroactive polymer element to a bony structure of the subject with the connecting element, wherein the electroactive polymer element is capable of modulating the opening of the air passageway. Another method is a method of treating a disease using an airway implant device by implanting an electroactive polymer element in a tongue of a subject and linking the electroactive polymer element to a jaw bone, the electroactive polymer element is capable of supporting the tongue when it is energized. The devices are used to treat sleeping disorders, such as obstructive sleep apnea or snoring.

One embodiment is an airway implant device having a electroactive polymer element and a connecting element, wherein the electroactive polymer element is capable of modulating the opening of an air passageway and the connecting element is used to fasten the electroactive polymer element to a rigid structure. Preferably, the rigid structure is a bony structure. In some embodiments, both the electroactive polymer element and connecting element are made from a polymeric material. The electroactive polymer element can include an ion-exchange polymer metal composite. In other embodiments, the electroactive polymer element can include a conducting polymer such as a polypyrrole, a carbon nanotube or a polyaniline.

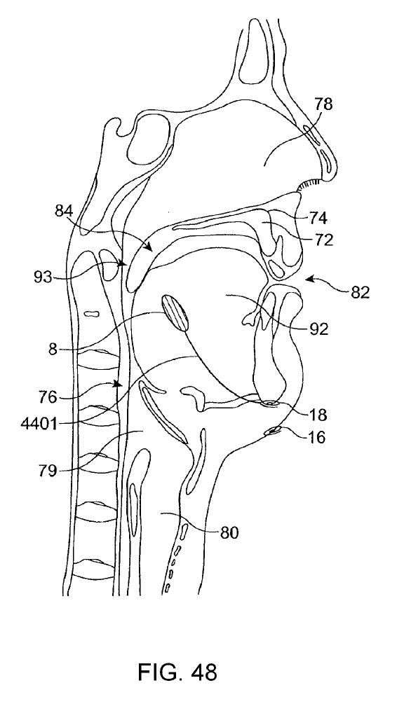

One embodiment of the airway implant device with a connecting element is depicted in FIG. 48. The electroactive polymer element 8 is linked to the jaw bone with a connecting element 4401. A first inductor 18 is implanted in the patient and a second inductor 16 is located on the outside and can be worn by the patient when the airway implant device needs to be activated, for example prior to going to sleep.

In another embodiment, the airway implant device with the connecting element further includes an anode, a cathode, a first inductor, and a controller. The anode and cathode are typically connected to the electroactive polymer element. The electroactive polymer element is energized with a power supply and is activated by electrical energy from the power supply. The electroactive polymer element can be physically connected to the power supply for example with a wire lead or can be connected with an inductive coupling mechanism.

The airway implant device with a connecting element further includes in some embodiments a non-implanted portion. Preferably the non-implanted portion is in the form of a strip and is used to control the electroactive polymer element. Typically this strip includes a power supply and a second inductor, the second inductor capable of interacting with a first inductor.

As set forth above, certain embodiments of the present invention are related to an implantable device for stabilizing the tongue during sleeping. P FIG. 49 is a simplified schematic drawing of an exemplary tongue implant device 5100 in accordance with another embodiment of the present invention. FIG. 49 is shown as a longitudinal sectional drawing to better show the interior of the implant 5100. The implant 5100 includes a bracket portion 5102 configured to be attached with the mandible. As is shown in FIG. 49 the bracket portion 5102 includes a plurality of apertures that render the bracket 5102 more flexible so as to be bent into a shape that is suitable for attaching the bracket 5102 with a patient's mandible. The bracket 5102 can be an off-the shelf titanium or stainless steel bracket that are non-magnetic in nature. A housing portion 5104 is connected at the distal end of the bracket 5102. The distal end of the deformable portion 5110 is connected with an anchor member 5112. The anchor 5112 need not be located at the distal end of the deformable portion 5110; it can be located at any length along the deformable portion. The anchor 5112 can be made from an absorbable material. The anchor 5112 is shown to have two sets of anchoring members 5113 and 5114. The distal anchoring member 5113 is configured to prevent an unintended insertion of the implant beyond the desired location, which could cause an exposure of the implant into the oral cavity. The anchor 5112 is also configured to be deployable using a suitable deployment sheath, such a deployment sheath having peal-away portions. Distal tip 5115 is configured to have a rounded and narrow shape to render the implant more easily deployable. Distal tip 5115 can be made of absorbable polymers like polylactic acid, polylacticglycolic acid, polysulfone, cellulose acetate, etc. In addition, the anchor 5112 and members 5113 and 5114 can be perforated members to help induce a fibrosis if need be.

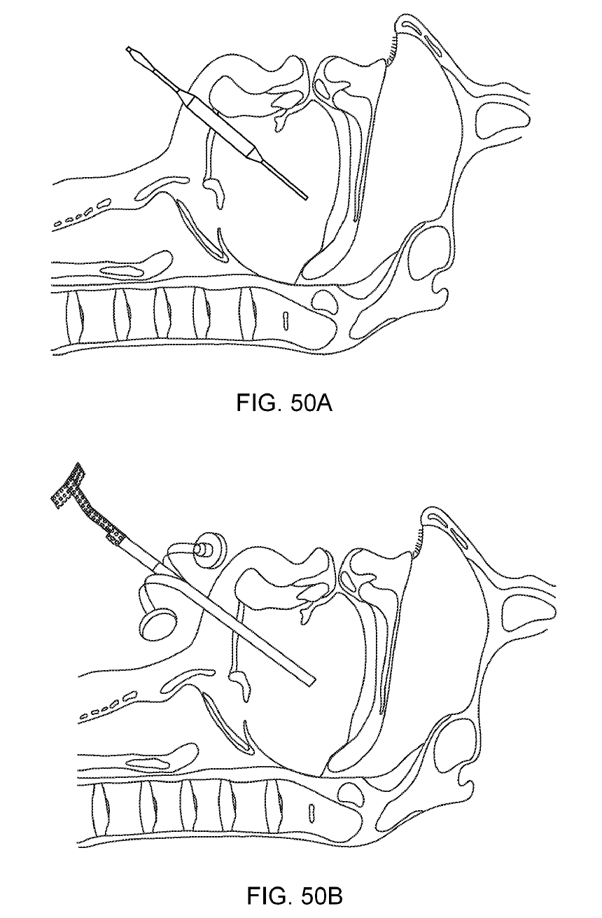

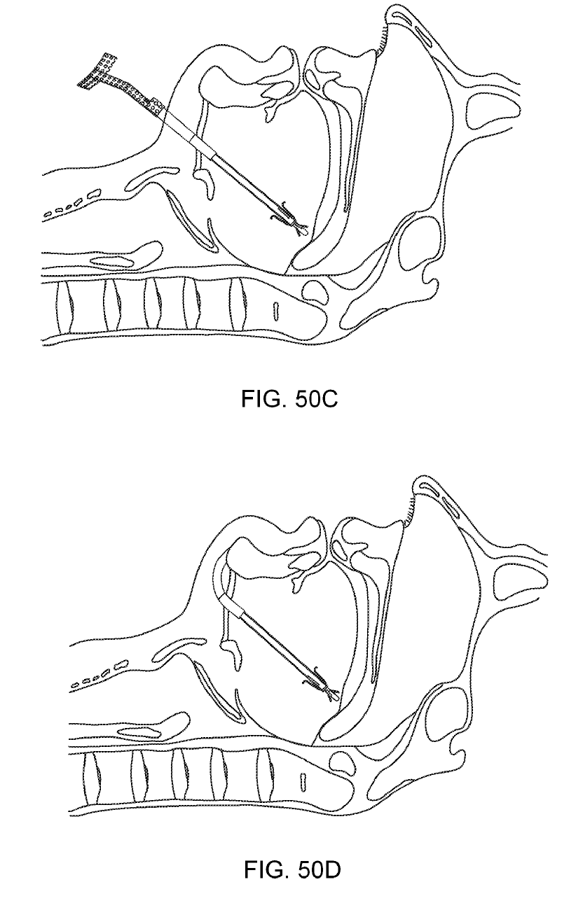

FIGS. 50A-D illustrate one exemplary procedure for the placement of the tongue implant. In FIG. 50A, tongue tissue is dissected to make room in the form of a tongue cavity for the implant. FIG. 50B shows that the implant along with a peal-away introducer is inserted into the created cavity. FIG. 50C shows that introducer is pulled back and away. The removal of the sheath deploys the implant. FIG. 50D. shows that in a last step, the bracket in the implant is anchored to the mandible.

### VII. Method of Using

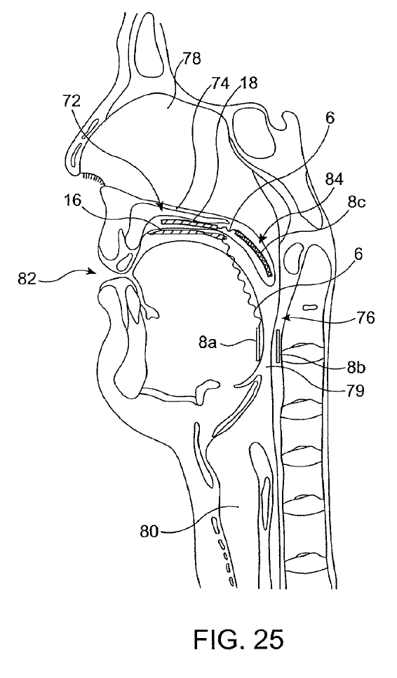

FIG. 25 illustrates an embodiment of a method of the airway implant device of the present invention. In this embodiment, the first inductor 18 is implanted in the mouth roof 72, for example in or adjacent to the hard palate 74. Wire leads 6 connect the first inductor 18 to the actuator elements 8a, 8b, and 8c. A first actuator element 8a is implanted in the base of the tongue at the pharynx wall 76. A second actuator element 8b is integral with the first actuator element 8a (e.g., as two sections of a hollow cylindrical actuator element 8, such as shown in FIG. 17). The first and second actuator elements 8a and 8b can be separate and unattached elements. The third actuator element 8c is implanted in the uvula and/or soft palate 84. The actuator elements 8 can also be implanted in the wall of the nasal passages 78, higher or lower in the pharynx 79, such as in the nasal pharynx, in the wall of the trachea 80, in the larynx (not shown), in any other airway, or combinations thereof. The second inductor 16 is worn by the patient in the mouth 82. The second inductor 16 is connected to an integral or non-integral power source. The second inductor 16 can be one or multiple induction coils. The second inductor 16 inductively transmits RF energy to the first inductor 18. The first inductor 18 changes the RF energy into electricity. The first inductor 18 sends a charge or current along the wire leads 6 to the actuator elements 8a, 8b, and 8c. The actuator elements 8a, 8b, and 8c are energized by the charge or current. The energized actuator elements 8a, 8b, and 8c increase the stiffness and/or alter the shape of the airways. The energized actuator elements 8a, 8b, and 8c modulate the opening of the airways around which the actuator elements 8a, 8b, and 8c are implanted. The non-energized actuator elements 8a, 8b, and 8c are configured to conform to the airway around which the actuator elements 8a, 8b, and 8c are implanted. The non-energized actuator elements 8a, 8b, and 8c are flexible and soft.

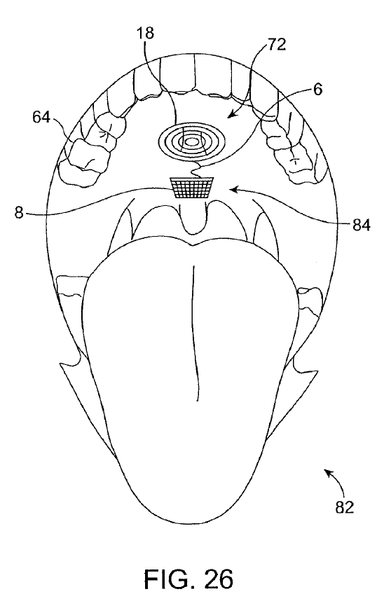

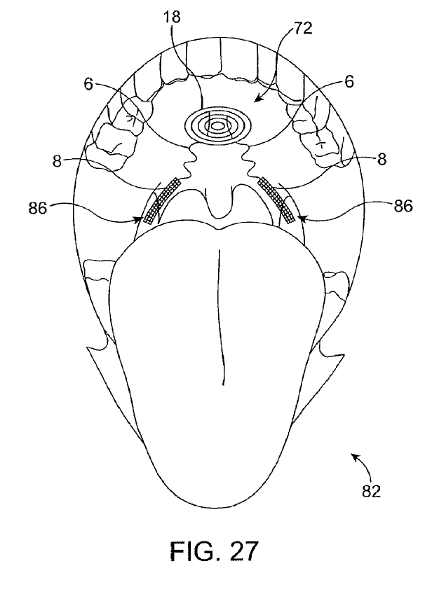

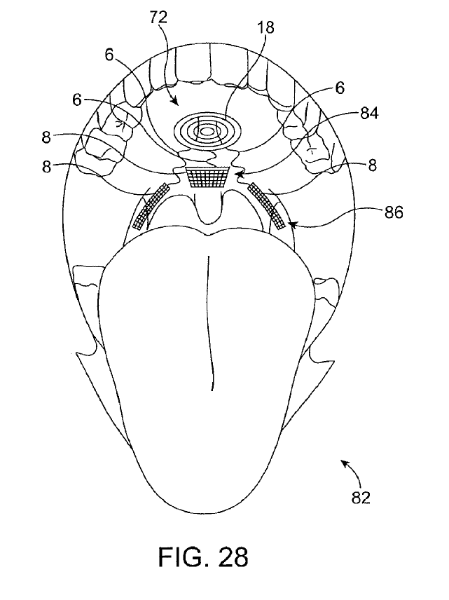

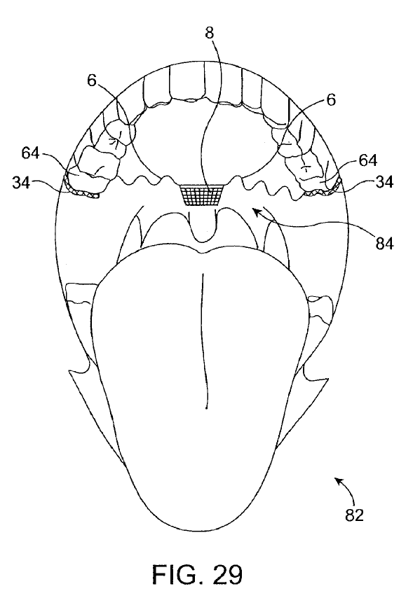

FIG. 26 illustrates another embodiment of the invention. In this embodiment, the first inductor 18 is implanted in the mouth roof 72 and attached to an actuator element 8 via the wire lead 6. The actuator element 8 is preferably in the soft palate 84. In another embodiment, FIG. 27 illustrates that the first inductor 18 is implanted in the mouth roof 72 and attached to two actuator elements 8 via two wire leads 6. The actuator elements 8 are implanted in side walls 86 of the mouth 82. In yet another embodiment, as illustrated in FIG. 28, the first inductor 18 is implanted in the mouth roof 72 and attached to three actuator elements 8 via three wire leads 6. The actuator elements 8 are implanted in the soft palate 84 and the side walls 86 of the mouth 82. FIG. 29 illustrates an embodiment in which the first conductors (not shown, e.g., the tooth sockets), are attached to, and in conductive electrical communication with, the second conductors. The mouthpiece 66, such as shown in FIG. 23, can be worn by the patient to energize the actuator element 8. The tooth sockets are removably attached to the first conductors 34. The first conductors 34 are dental fillings, conductive posts adjacent to and/or through the teeth 64.

FIG. 33 illustrates an embodiment in which a patient 88 has the first receiver (not shown) implanted in the patient's cheek and wears the non-implanted portion 22, such as shown in FIG. 24, on the outside of the patient's cheek. The non-implanted portion 22 energizes the implanted portion (not shown).

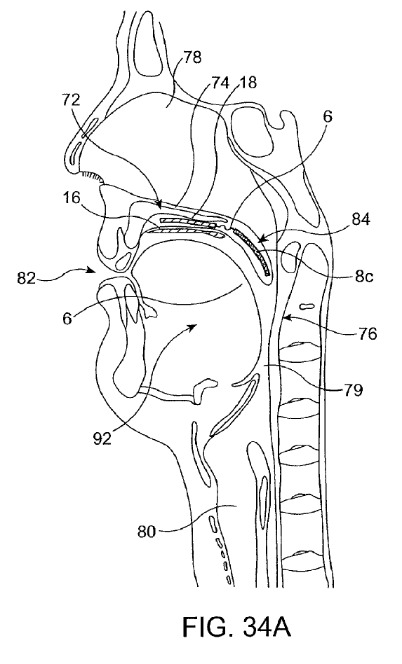

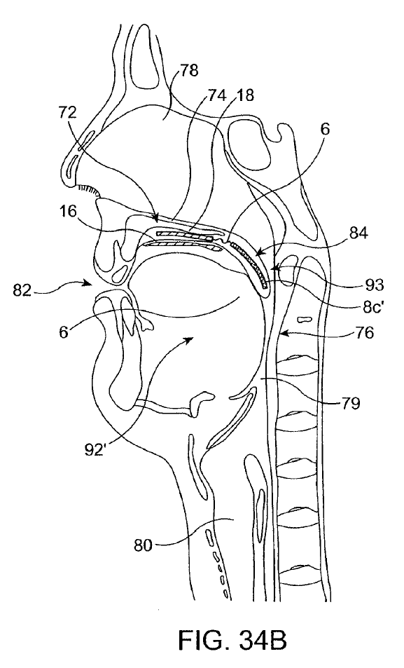

FIGS. 34-36 depict some of the ways in which the implant devices function to open the airways. FIGS. 34A and 34B depict a side view of a patient with a soft palate implant 8c and a non-implanted portion of the device, with a second inductor 16, which in this case is a wearable mouth piece. The wearable mouth piece includes a transmitter coil, a power source, and other electronics, which are not depicted. Also, shown is a first inductor 18. The implant device has the ability to sense and deflect the tongue so as to open the airway. FIG. 34A depicts the tongue 92 in its normal state. During sleep, when the tongue collapses 92′, as shown in FIG. 34B, the actuator element 8c′ senses the collapsed tongue and is energized via the mouthpiece and first inductor and it stiffens to push away the tongue from the airway and keeps the airway open. This opening of the airway can be partial or complete. In some embodiments, particularly the embodiments without the sensor, the implant is powered when the patient is asleep such that the actuator element 8 is energized and keeps the collapsed tongue away from the airway.

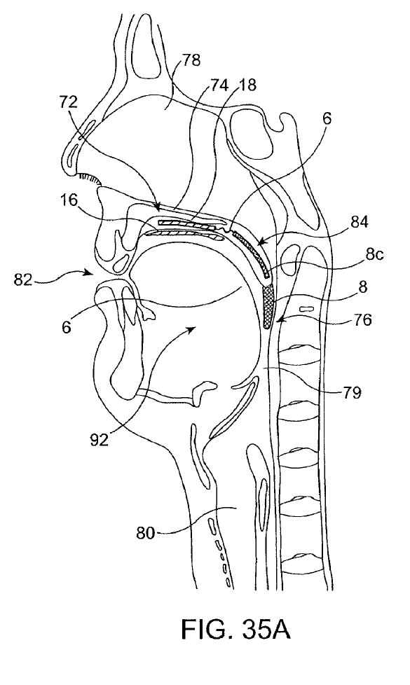

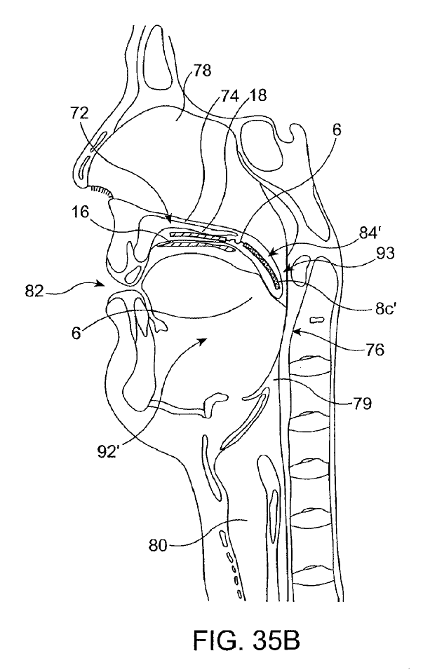

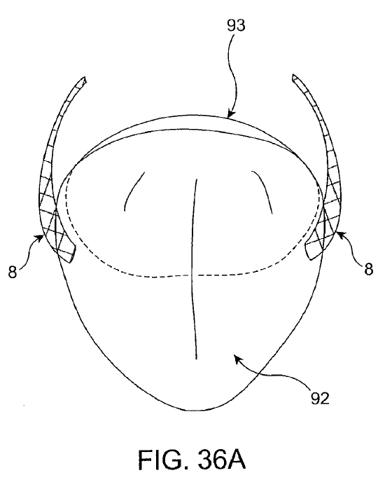

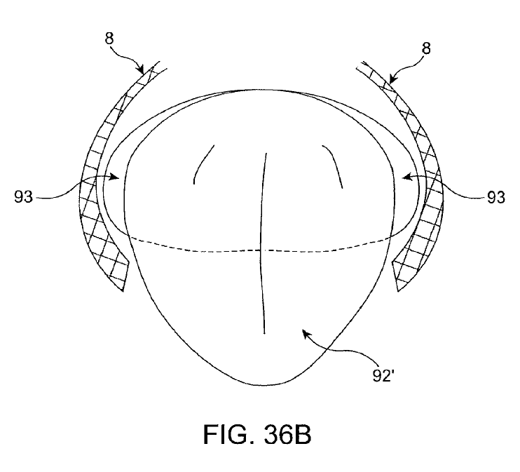

FIGS. 35 and 36 depict an embodiment of keeping the airways open with lateral wall implants. FIG. 35A shows a side view of a patient's face with an actuator element 8 located in the lateral wall of the airway. FIG. 35A depicts the tongue 92 in its normal state. FIG. 35B depicts the tongue 92′ in a collapsed state. When the tongue is in this state or before it goes into the collapsed state the actuator element 8 is energized so as to stretch the lateral walls and open the airway, as shown in FIG. 36B. FIGS. 36A and 36B are a view of the airway as seen through the mouth of patient. FIG. 36A depicts the actuator elements 8 in a non-energized state and the tongue in a non-collapsed state. When the tongue collapses or it has a tendency to collapse, such as during sleep, the actuator element 8 is energized and airway walls are pushed away from the tongue and creates an open air passageway 93. This embodiment is particularly useful in obese patients.

### VIII. Airway Diseases

During sleep, the muscles in the roof of the mouth (soft palate), tongue and throat relax. If the tissues in the throat relax enough, they vibrate and may partially obstruct the airway. The more narrowed the airway, the more forceful the airflow becomes. Tissue vibration increases, and snoring grows louder. Having a low, thick soft palate or enlarged tonsils or tissues in the back of the throat (adenoids) can narrow the airway. Likewise, if the triangular piece of tissue hanging from the soft palate (uvula) is elongated, airflow can be obstructed and vibration increased. Being overweight contributes to narrowing of throat tissues. Chronic nasal congestion or a crooked partition between the nostrils (deviated nasal septum) may be to blame.

Snoring may also be associated with sleep apnea. In this serious condition, excessive sagging of throat tissues causes your airway to collapse, preventing breathing. Sleep apnea generally breaks up loud snoring with 10 seconds or more of silence. Eventually, the lack of oxygen and an increase in carbon dioxide signal causes the person to wake up, forcing the airway open with a loud snort.

Obstructive sleep apnea occurs when the muscles in the back of the throat relax. These muscles support the soft palate, uvula, tonsils and tongue. When the muscles relax, the airway is narrowed or closed during breathing in, and breathing is momentarily cut off. This lowers the level of oxygen in the blood. The brain senses this decrease and briefly rouses the person from sleep so that the airway can be reopened. Typically, this awakening is so brief that it cannot be remembered. Central sleep apnea, which is far less common, occurs when the brain fails to transmit signals to the breathing muscles.

Thus, it can be seen that airway disorders, such as sleep apnea and snoring, are caused by improper opening of the airway passageways. The devices and methods described herein are suitable for the treatment of disorders caused by the improper opening of the air passageways. The devices can be implanted in any suitable location such as to open up the airways. The opening of the passageways need not be a complete opening and in some conditions a partial opening is sufficient to treat the disorder.

In addition to air passageway disorders, the implants disclosed herein are suitable for use in other disorders. The disorders treated with the devices include those that are caused by improper opening and/or closing of passageways in the body, such as various locations of the gastro-intestinal tract or blood vessels. The implantation of the devices are suitable for supporting walls of passageways The devices can be implanted in the walls of the gastro-intestinal tract, such as the esophagus to treat acid reflux. The gastro-intestinal tract or blood vessel devices can be used in combination with the sensors described above. Also, the implants and/or sphincters can be used for disorders of fecal and urinary sphincters. Further, the implants of the invention can be tailored for specific patient needs. It is apparent to one skilled in the art that various changes and modifications can be made to this disclosure, and equivalents employed, without departing from the spirit and scope of the invention. Elements shown with any embodiment are exemplary for the specific embodiment and can be used on other embodiments within this disclosure.

### IX. Device Testing

The devices of the present invention are tested for mechanical fatigue and electromechanical life cycle. Mechanical fatigue is tested using a piston clamped to one end of the test sample and the other end (corresponding to the hard palatal portion) of the sample is fixed by a clamp. The frequency of the piston movement is set at 2 Hz and bending angle is set at −75°. The fatigue data records the number of times the piston moves up and down before the conductive polymer layer cracks. The tests were performed at room temperature and dry state, which is expected to represent the worst case scenario for the stability of the conductive polymer layer.

Using the test above, the sample with the silicone rubber coating is capable of withstanding more than 2 million cycles at 2.2 Hz over 10 days. This corresponds to a lifespan of greater than 5 years. The control device without the silicone rubber coating lasted less than 10 cycles, with an equivalent lifespan of less than 1 month. In addition, the device using a patch coated conductive polymer in a staggered design configuration (see FIG. 53) can withstand at least 3 million cycles, with an equivalent lifespan of more than 8 years.

Electromechanical life cycle was also tested using several means. In one test, samples were cycled between −1.2 to +1.2V by using 1.2V for 1 minute, followed by 2 minute rest and then application of negative 1.2V for 1 minute and resting for 2 minutes. The sample with silicone coating showed more than two times charge capacity than the control sample during the first 400 cycles and gradually decreased the charge capacity similar to that of the controls (FIG. 54).

In another test of electromechanical strength, the sample was actuated using 1.2V and 40 uAhr capacity followed by 8 hour holding. The longer the actuation time, the higher the sample impedance. When the actuation time is higher than 1000 seconds, the sample reached the end of the sample's life cycle. With the silicone coating, sample life cycle was more than 30 cycles while the control samples showed less than 20 cycles (FIG. 55).

As will be understood by those skilled in the art, the present invention may be embodied in other specific forms without departing from the essential characteristics thereof. Those skilled in the art will recognize, or be able to ascertain using no more than routine experimentation, many equivalents to the specific embodiments of the invention described herein. Such equivalents are intended to be encompassed by the following claims.

What is claimed is:

1. An airway implant device comprising an electroactive polymer element, comprising:
a composite layer comprising a polymer substrate and a biocompatible conductive material, wherein the composite layer further comprises opposing surfaces; and
a conductive polymer layer disposed on at least one of the opposing surfaces of the composite layer, wherein the implant device is adapted and configured to modulate an opening of an air passageway.

2. The device of claim 1, wherein the polymer substrate comprises a material selected from the group consisting of polytetrafluoroethylene, polyfluorosulfonic acid, perfluorosulfonate, polyvinylidene fluoride, polyethylene, polypropylene, polystyrene, polyaniline, polyacrylonitrile, cellulose, regenerated cellulose, cellulose acetate, polysulfone, polyurethane, polyvinyl alcohol, polyvinyl acetate, polyvinyl pyrrolidone, polymethyl methacrylate, silicon and combinations thereof.

3. The device of claim 1, wherein the polymer substrate comprises polyurethane.

4. The device of claim 1, wherein the biocompatible conductive material is selected from the group consisting of conductive carbon, Ag, Au, Cu, Pt, Pd, Rh, Ir, Ru, Os and Re.

5. The device of claim 1, wherein the biocompatible conductive material comprises Pt.

6. The device of claim 1, wherein the polymer substrate is coated with the biocompatible conductive material.

7. The device of claim 1, wherein the polymer substrate is embedded with the biocompatible conductive material, wherein the ratio of biocompatible conductive material to polymer substrate is from about 0.1:1 (w/w) to about 5:1 (w/w).

8. The device of claim 7, wherein the ratio of biocompatible conductive material to polymer substrate is about 2:1 (w/w).

9. The device of claim 1, wherein the biocompatible conductive material is in the form of wires or particles.

10. The device of claim 9, wherein the wires have a pitch of from about 1 μm to about 1 mm.

11. The device of claim 9, wherein the particles are from about 0.1 μm to about 100 μm in size.

12. The device of claim 1, wherein the conductive polymer layer comprises a polymer selected from the group consisting of polypyrrole, polyaniline and polyacetylene.

13. The device of claim 1, wherein the conductive polymer layer comprises polypyrrole.

14. The device of claim 1, wherein the conductive polymer layer comprises a copolymer.

15. The device of claim 14, wherein the copolymer comprises pyrrole and N-methylpyrrole.

16. The device of claim 1, wherein the conductive polymer layer further comprises a dopant.

17. The device of claim 16, wherein the dopant comprises an ionic dopant.

18. The device of claim 17, wherein the ionic dopant comprises a biocompatible ionic dopant.

19. The device of claim 1, wherein the biocompatible ionic dopant comprises Na⁺.

20. The device of claim 16, wherein the dopant comprises dodecyl benzenesulfonic acid.

21. The device of claim 1, wherein the composite layer comprises polyurethane and Pt.

22. The device of claim 1, wherein the conductive polymer layer comprises polypyrrole doped with dodecyl benzenesulfonic acid.

23. The device of claim 1, wherein the conductive polymer layer comprises a copolymer of pyrrole and N-methylpyrrole doped with dodecyl benzenesulfonic acid.

24. The device of claim 1, wherein the electroactive polymer element comprises a composite of polyurethane and Pt, and polypyrrole doped with dodecyl benzenesulfonic acid.

25. The device of claim 1, wherein the electroactive polymer element comprises two conductive polymer layers each deposited on one of the opposing faces of the composite layer.

26. The device of claim 25, wherein at least one of the opposing surfaces is patch coated with one of the conductive polymer layers.

27. The device of claim 25, wherein both of the opposing surfaces are patch coated with the conductive polymer layers.

28. The device of claim 1, comprising a plurality of composite layers and a plurality of conductive polymer layers in alternating layers such that each conductive polymer layer is disposed on an opposing face of one of the composite layers.

29. The device of claim 1, further comprising a silicone rubber coating.

30. The device of claim 1, further comprising an anode, a cathode, a first inductor, a controller and a non-implanted portion.

31. The device of claim 30, wherein the non-implanted portion comprises a mouthguard, a power supply and a second inductor.

32. The device of claim 31, wherein the first inductor and the second inductor are configured to interact.

33. The device of claim 30, wherein the electroactive polymer element further comprises wires for connection with the first inductor.

34. The device of claim 1, wherein the electroactive polymer element is configured for implantation into a soft palate, a lateral pharyngeal wall, a tongue or combination thereof.

35. The device of claim 1, wherein the device further comprises a coating to prevent or promote tissue growth.

36. The device of claim 35, wherein the device further comprises a coating selected from the group consisting of polypropylene, poly-L-lysine, poly-D-lysine, polyethylene glycol, polyvinyl alcohol, polyvinyl acetate, polymethyl methacrylate, hyaluronic acid and combinations thereof.

37. The device of claim 1, wherein the airway implant device is controlled by an inductive coupling mechanism.

38. The device of claim 1, wherein the electroactive polymer element further comprises a sodium source disposed on one of the opposing surfaces of the composite layer.

39. A method of controlling an opening of an air passageway, comprising:
implanting an airway implant device proximal to an air passageway, in a wall of an air passageway or in both, the device comprising an electroactive polymer element comprising:
a composite layer comprising a polymer substrate and a biocompatible conductive material, wherein the composite layer further comprises opposing surfaces; and
a conductive polymer layer disposed on at least one of the opposing surfaces of the composite layer, wherein the implant device is adapted and configured to modulate an opening of an air passageway; and
energizing the electroactive polymer element for a fixed period of time, such that the electroactive polymer element adopts an energized state and maintains the energized state after the fixed period of time has passed, thereby completely or partially opening the air passageway.

40. The method of claim 39, further comprising de-energizing the electroactive polymer element to a non-energized state.

41. The method of claim 39, wherein the implantation of the airway implant device is in a soft palate, a lateral pharyngeal wall, a tongue or a combination thereof.

42. The method of claim 39, wherein the airway implant device is controlled by an inductive coupling mechanism.

43. A method of treating a disease using an airway implant device, comprising:
implanting an airway implant device proximal to an air passageway or in a wall of an air passageway or in both, the device comprising an electroactive polymer element comprising:
a composite layer comprising a polymer substrate and a biocompatible conductive material, wherein the composite layer further comprises opposing surfaces; and
a conductive polymer layer disposed on at least one of the opposing surfaces of the composite layer, wherein the implant device is adapted and configured to modulate an opening of an air passageway; and
energizing the electroactive polymer element for a fixed period of time, such that the electroactive polymer element adopts an energized state and maintains the energized state after the fixed period of time has passed, thereby treating the disease.

44. The method of claim 43, wherein the disease is obstructive sleep apnea and/or snoring.

45. The method of claim 43, wherein the airway implant device is controlled by an inductive coupling mechanism.

46. The method of claim 43, wherein the airway implant device is implanted in a soft palate, and the energizing of the electroactive polymer element supports the soft palate.

47. The method of claim 43, wherein the airway implant device is implanted in a lateral pharyngeal wall, and the energizing of the electroactive polymer element prevents the lateral pharyngeal wall from collapsing.

48. The method of claim 43, wherein the airway implant device is implanted in a tongue, and the energizing of the electroactive polymer element prevents the tongue from collapsing.
